(19) United States
(12) Patent Application Publication
(10) Pub. No.: US 2026/0097185 A1
(43) Pub. Date: Apr. 9, 2026

(54) SYSTEMS AND METHODS FOR VARYING BENDING STIFFNESS OF A FLEXIBLE ELONGATE DEVICE

(71) Applicant: INTUITIVE SURGICAL OPERATIONS, INC., Sunnyvale, CA (US)

(72) Inventors: Rumen Deyanov, Fremont, CA (US); Russell Field, Pacifica, CA (US)

(21) Appl. No.: 19/112,852

(22) PCT Filed: Sep. 18, 2023
(86) PCT No.: PCT/US2023/074426

§ 371 Date: Mar. 18, 2025

Related U.S. Application Data
(60) Provisional application No. 63/407,899, filed on Sep. 19, 2022.
(60) Provisional application No. 63/407,941, filed on Sep. 19, 2022.

(51) Int. Cl. A61M 25/01; A61B 34/30; A61M 25/00

(52) U.S. Cl. CPC A61M 25/0141; A61M 25/0045; A61M 25/0113; A61M 25/0138; A61B 2034/301; A61M 25/0009

(57) ABSTRACT

A flexible elongate device comprises an articulable distal section and a non-articulable proximal section. The non-articulable proximal section is proximal to the articulable distal section. A distal portion of the non-articulable proximal section has a first bending stiffness, and a proximal portion of the non-articulable proximal section has a second bending stiffness. The first bending stiffness is less than the second bending stiffness.

### CROSS-REFERENCED APPLICATIONS

This application claims priority to and the benefit of U.S. Provisional Application No. 63/407,899, filed Sep. 19, 2022, entitled “Systems and Methods for Varying Bending Stiffness of a Flexible Elongate Device,” and also claims priority to and the benefit of U.S. Provisional Application No. 63/407,941, filed Sep. 19, 2022, entitled “Systems and Methods for Varying Bending Stiffness of a Flexible Elongate Device,” each of which is incorporated by reference herein in its entirety.

### FIELD

Examples described herein relate to systems and methods for varying bending stiffness of a flexible elongate device. In particular, examples described herein relate to systems and methods of varying a ribbon width of a coil of a flexible elongate device and/or bonding layers of the flexible elongate device to vary the bending stiffness of the flexible elongate device.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, an operator may insert minimally invasive medical tools to reach a target tissue location. Minimally invasive medical tools include instruments such as therapeutic, diagnostic, biopsy, and surgical instruments, as well as flexible elongate devices having lumens that hold these instruments and traverse anatomical passageways to guide the instruments to the target tissue location.

### SUMMARY

Various features may allow a flexible elongate device, such as a catheter, to have variable bending stiffness along the length of the flexible elongate device. The following presents a summary of various examples described herein and is not intended to identify key or critical elements or to delineate the scope of the claims.

Consistent with some examples, a flexible elongate device is provided. The flexible elongate device includes an articulable distal section and a non-articulable proximal section that is proximal to the articulable distal section. A distal portion of the non-articulable proximal section has a first bending stiffness and a proximal portion of the non-articulable proximal section has a second bending stiffness. The first bending stiffness is less than the second bending stiffness.

Consistent with other examples, a flexible elongate device is provided. The flexible elongate device includes a coil layer that provides variable bending stiffness for the flexible elongate device along an axial length of the coil layer. The coil layer includes a plurality of coils, and the plurality of coils include a first coil including ribbon elements having variable ribbon width. In a first region along the axial length of the coil layer having a first bending stiffness, a first ribbon element of the first coil has a first ribbon width. In a second region along the axial length of the coil layer having a second bending stiffness that is higher than the first bending stiffness. A second ribbon element of the first coil has a second ribbon width that is wider than the first ribbon width.

Consistent with other examples, a flexible elongate device is provided. The flexible elongate device includes a coil layer that provides variable bending stiffness for the flexible elongate device along an axial length of the coil layer. The coil layer includes a plurality of coils, and the plurality of coils includes a first coil including ribbon elements having variable ribbon width. In a first region along the axial length of the coil layer having a first bending stiffness, a first ribbon element of the first coil has a first ribbon width. In a second region along the axial length of the coil layer having a second bending stiffness that is higher than the first bending stiffness. A second ribbon element of the first coil has a second ribbon width that is wider than the first ribbon width. The plurality of coils further includes a second coil and a third coil. Adjacent coils of the plurality of coils have opposite pitch directions.

Consistent with other examples, a flexible elongate device is provided. The flexible elongate device includes a coil layer that provides variable bending stiffness for the flexible elongate device along an axial length of the coil layer. The coil layer includes an outer coil, an inner coil within the outer coil, and bonds between the inner coil and the outer coil that couple the inner and outer coils to each other. In a first region along the axial length of the coil layer having a first bending stiffness, a first subset of the bonds have a first spatial density. In a second region along the axial length of the coil layer having a second bending stiffness that is higher than the first bending stiffness, a second subset of the bonds have a second spatial density that is higher than the first spatial density.

Consistent with other examples, a method is provided. The method includes constructing a first coil and a second coil. The first coil includes ribbon elements having variable ribbon width. The method further includes constructing a coil layer using the first coil and the second coil and bonding the first coil to the second coil. In a first region along an axial length of the coil layer having a first bending stiffness, a first ribbon element of the first coil has a first ribbon width, and in a second region along the axial length of the coil layer having a second bending stiffness that is higher than the first bending stiffness. A second ribbon element of the first coil has a second ribbon width that is wider than the first ribbon width.

Consistent with other examples, a method is provided. The method includes positioning a coil layer of a flexible elongate device around a conductive mandrel. The coil layer includes an outer coil and an inner coil within the outer coil. The method further includes guiding the coil layer between a plurality of conductive wheels. The conductive wheels are configured to contact the coil layer. The method further includes welding the outer coil to the inner coil with at a plurality of welding locations by applying an electric current between the mandrel and the conductive wheels. In a first region along an axial length of the coil layer having a first bending stiffness, a first subset of welding locations of the plurality of welding locations has a first spatial density. In a second region along the axial length of the coil layer having a second bending stiffness that is higher than the first bending stiffness, a second subset of welding locations of the plurality of welding locations has a second spatial density that is higher than the first spatial density.

Other examples include corresponding computer systems, apparatus, and computer programs recorded on one or more computer storage devices, each configured to perform the actions of any one or more methods described below.

It is to be understood that both the foregoing general description and the following detailed description are illustrative and explanatory in nature and are intended to provide an understanding of the various examples described herein without limiting the scope of the various examples described herein. In that regard, additional aspects, features, and advantages of the various examples described herein will be apparent to one skilled in the art from the following detailed description.

### BRIEF DESCRIPTIONS OF THE DRAWINGS

FIG. 1A is a cross-sectional front view of a flexible elongate device including a coil layer according to some examples.

FIG. 1B is a cross-sectional side view of the flexible elongate device shown in FIG. 1A according to some examples.

FIG. 2A is a side view of an outer coil, an intermediate coil, and an inner coil of a coil layer of a flexible elongate device according to some examples.

FIG. 2B is a side view of the inner coil shown in FIG. 2A with variable ribbon width along a length of a coil layer of a flexible elongate device according to some examples.

FIG. 3 is a side view of a flexible elongate device with a non-articulable proximal section and an articulable distal section according to some examples.

FIG. 4 illustrates a portion of a coil layer of a flexible elongate device according to some examples.

FIGS. 5A and 5B illustrate a portion of a coil layer of a flexible elongate device with various coil configurations according to some examples.

FIG. 6 is a flowchart illustrating a method of manufacturing a coil layer of a flexible elongate device according to some examples.

FIG. 7A is a side view of a coil layer of a flexible elongate device with axially variably spaced bonding locations according to some examples.

FIG. 7B is a side view of an outer coil and an inner coil of a coil layer of a flexible elongate device with axially variably spaced bonding locations according to some examples.

FIG. 7C is a side view of a distal portion of a coil layer of a flexible elongate device with axially variably spaced bonding locations according to some examples.

FIG. 7D is a cross-sectional front view of a coil layer of a flexible elongate device with radially variably spaced bonding locations according to some examples.

FIG. 8 is a side view of a flexible elongate device with a non-articulable proximal section and an articulable distal section according to some examples.

FIGS. 9A-9C illustrate a distal portion of a coil layer of a flexible elongate device with various coil winding patterns according to some examples.

FIG. 10 illustrates a welding mechanism for a flexible elongate device according to some examples.

FIG. 11 is a flowchart illustrating a method of welding a coil layer of a flexible elongate device according to some examples.

FIG. 12 is a diagram of a teleoperated medical system according to some examples.

FIG. 13 is a diagram of a medical instrument system according to some examples.

Various examples described herein and their advantages are described in the detailed description that follows. It should be appreciated that like reference numerals are used to identify like elements illustrated in one or more of the figures for purposes of illustrating but not limiting the various examples described herein.

### DETAILED DESCRIPTION

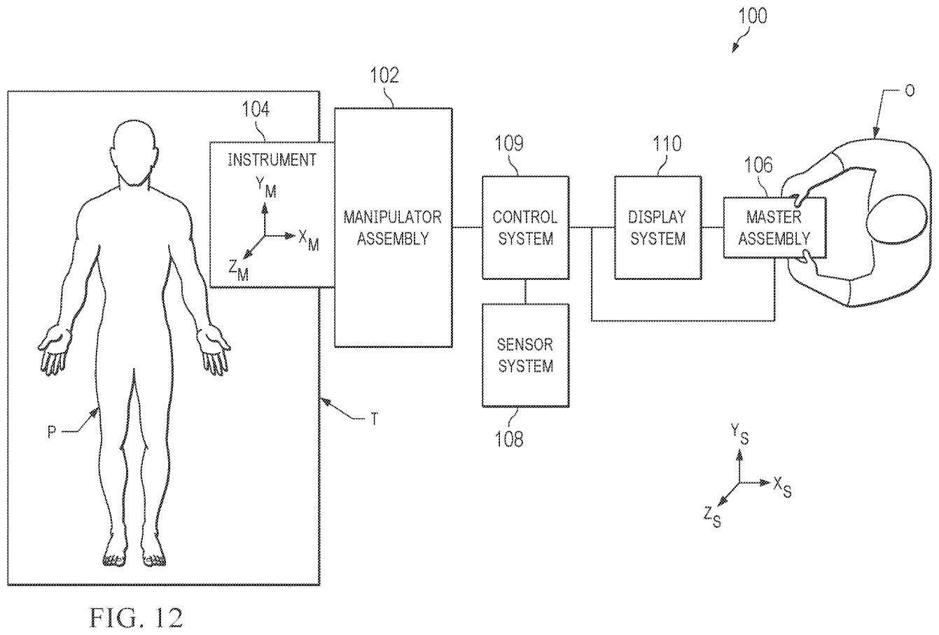

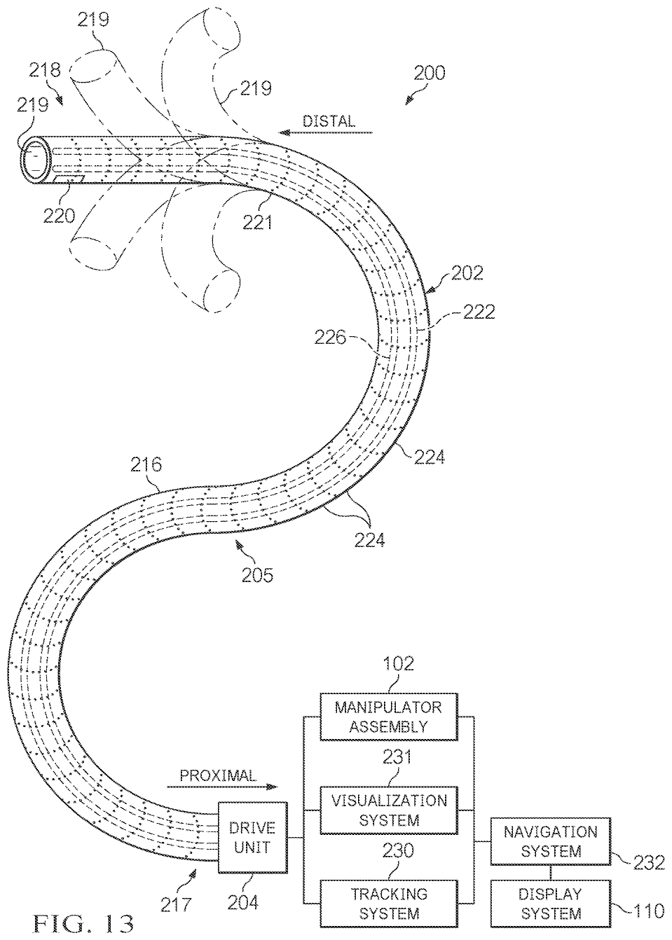

The present disclosure describes flexible elongate devices that may be used, for example, in medical systems to provide variable bending stiffness along the length of the flexible elongate devices. Examples of flexible elongate devices may include a catheter, a medical instrument, a sheath, or any other type of device that includes a flexible elongate body. Examples of medical systems that may incorporate any of the flexible elongate devices described herein are provided at FIG. 12, which shows a medical system 100, and FIG. 13, which shows a medical instrument system 200.

A flexible elongate device, such as a catheter, an instrument, a sheath, or any other flexible elongate body, may have variable bending stiffness along an axial length of the flexible elongate device. The flexible elongate device includes a coil layer that may assist in maintaining the patency of a lumen of the flexible elongate device (and any other lumens) during articulation and navigation of the flexible elongate device. The coil layer may also provide flexibility to the flexible elongate device, while allowing for variable bending stiffness of the flexible elongate device. For example, the coil layer includes variable ribbons to provide more bending stiffness in one area versus the other hence achieving a variation in bending stiffness along the length of the shaft. The coil layer may also provide structural support for the flexible elongate device. The coil layer may include at least two layers of coils, such as three layers of coils in one example. Each coil may include ribbon elements that form the coil structure. The ribbon elements of at least one of the coils may have a variable ribbon width along an axial length of the coil layer to provide for the variable bending stiffness. In general, a region of a coil having larger ribbon widths has higher bending stiffness and a region of the coil having smaller ribbon widths has a lower bending stiffness. One or more other coils of the coil layer may have ribbon elements with a consistent ribbon width along the axial length of the coil layer.

Additionally or alternatively, the coils may be bonded together at select, variably spaced locations along an axial length of the coil layer. The variably spaced bonds allow the flexible elongate device to have variable bending stiffness that may be tuned to a customized bending stiffness profile by customizing the locations where the layers of coils are bonded. The bending stiffness may be varied along the axial length of the coil layer, for example, when the bonds are at bonding locations with varied spacing along the axial length of the coil layer. The bending stiffness of the flexible elongate device may be varied around the radial length of the coil layer when the bonds are at bonding locations with varied spacing around the circumference of the coil layer. Customizing the bending stiffness profile of the flexible elongate device may obviate occurrences of the flexible elongate device being bent to a maximum bending stiffness or beyond a maximum bending stiffness. This may extend the longevity of the flexible elongate device and may result in the flexible elongate device being more responsive to movement commands during a medical procedure. Additionally, customizing the bending stiffness profile for the flexible elongate device may allow the bending stiffness of the flexible elongate device to be tuned to achieve a more optimized performance as a bendable flexible elongate device, where the performance depends on the bending stiffness parameters of the flexible elongate device.

In one example, the coil layer includes three coils. Adjacent coils of the coil layer have opposing pitch directions. One coil of the coil layer, such as the inner coil of the coil layer, includes ribbon elements with variable ribbon width along the axial length of the coil layer. The outer coil and the intermediate coil are each closed pitch. The inner coil is open pitch. The three coils are bonded (e.g., welded) at the proximal and distal ends of the coil layer. The three coils are each made of stainless steel but could alternatively be made of other metal or plastic materials suitable for coiling.

In some examples, one of the coils of the coil layer may have ribbon elements with variable ribbon width. This allows the flexible elongate device to have high bi-directional torsional stiffness and high axial stiffness in both compression and tension. Varying the bending stiffness of the flexible elongate device as a result of a coil layer with variable ribbon width may obviate occurrences of the flexible elongate device being bent to a maximum bending stiffness, beyond a maximum bending stiffness, or to small radii or beyond a minimum bend radius that damages the flexible elongate device. This may extend the longevity of the flexible elongate device and may result in the flexible elongate device being more responsive to movement commands during a medical procedure. Having a coil with variable ribbon width may allow the flexible elongate device to have resistance to ovalization, resistance to kinking and plastic deformation, while maintaining a thin wall, which may reduce the overall outer diameter of the flexible elongate device. In some examples, the coil layer includes multiple layers of coils (e.g., three layers of coils). A coil layer with multiple layers of coils, such as three layers of coils, may have higher torsional and axial stiffness than a coil layer with fewer layers of coils, such as one or two layers of coils.

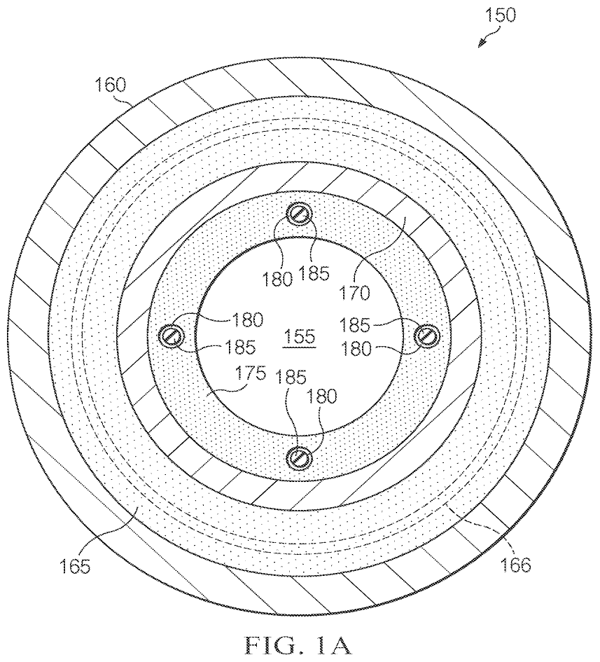

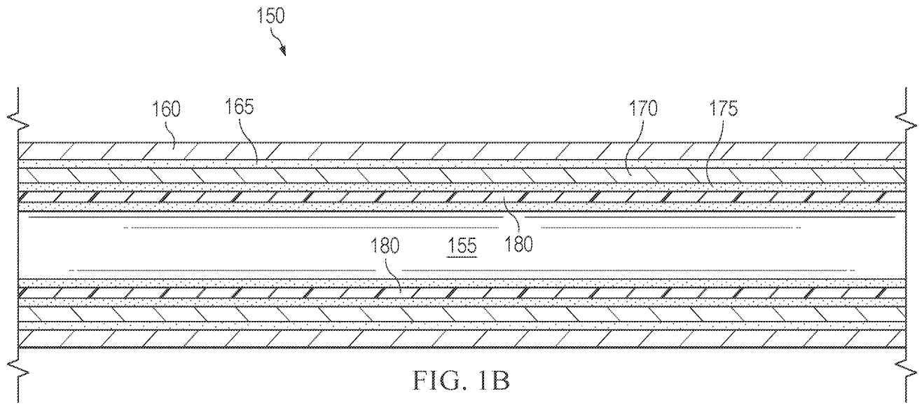

FIGS. 1A and 1B illustrate a cross-sectional front view and a cross-sectional side view, respectively, of a flexible elongate device 150 (also referred to as “elongate device 150”). The elongate device 150 may include a multi-layered, hollow cylindrical tube defining a lumen 155. The elongate device 150 may include one or more articulable sections and one or more non-articulable sections. In one example, the elongate device 150 includes an articulable distal section and a non-articulable proximal section that is proximal to the articulable distal section. In FIGS. 1A and 1B, a non-articulable section is shown. The elongate device 150 at a non-articulable section may include an outer sheath 160, a support layer 165, a coil layer 170, and an inner sheath 175, each of which is disposed concentrically and co-axially about the lumen 155. In some examples, the lumen 155 is a working lumen sized to receive one or more devices, such as a tool (e.g., ablation probe, biopsy needle, etc.), a sensor (e.g., a vision probe, ultrasound probe, etc.), or another articulable flexible elongate device (e.g., a catheter). In some examples, a conduit (not shown), which may define the lumen 155, is within the inner sheath 175. In some examples, the elongate device 150 may include multiple lumens (e.g., within the inner sheath 175).

The outer sheath 160 may include a length of flexible tubing extending from an inner surface to an outer surface. The outer sheath 160 may provide protection to the more inner components of the elongate device 150 and may assist in maintaining the patency of the lumen 155 (and any other lumens) during articulation or navigation of the elongate device 150. The outer sheath 160 may be metal, polymer, a composite, or any other suitable material.

The support layer 165 may include an embedded support component 166, which assists in maintaining the patency of the lumen 155 (and any other lumens) during articulation or navigation of the elongate device 150. The support layer 165 may be metal, polymer, a composite, or any other suitable material. In some examples, the support component 166 is a tubular braided element such as, by way of non-limiting example, a polyimide braid. The support component 166 may resist radial expansion and/or increase torsional stiffness. The support component 166 is sandwiched within the support layer 165, which may be fabricated of two separately extruded lengths of flexible tubing that may be bonded to one another and/or the support component 166. Other examples may lack a support layer 165 and/or the support component 166.

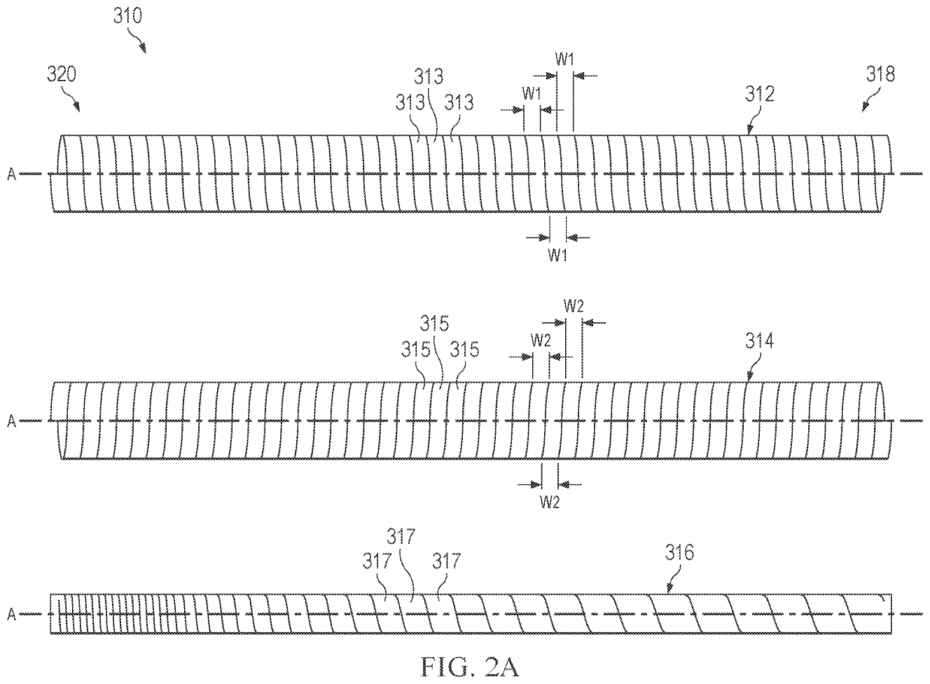

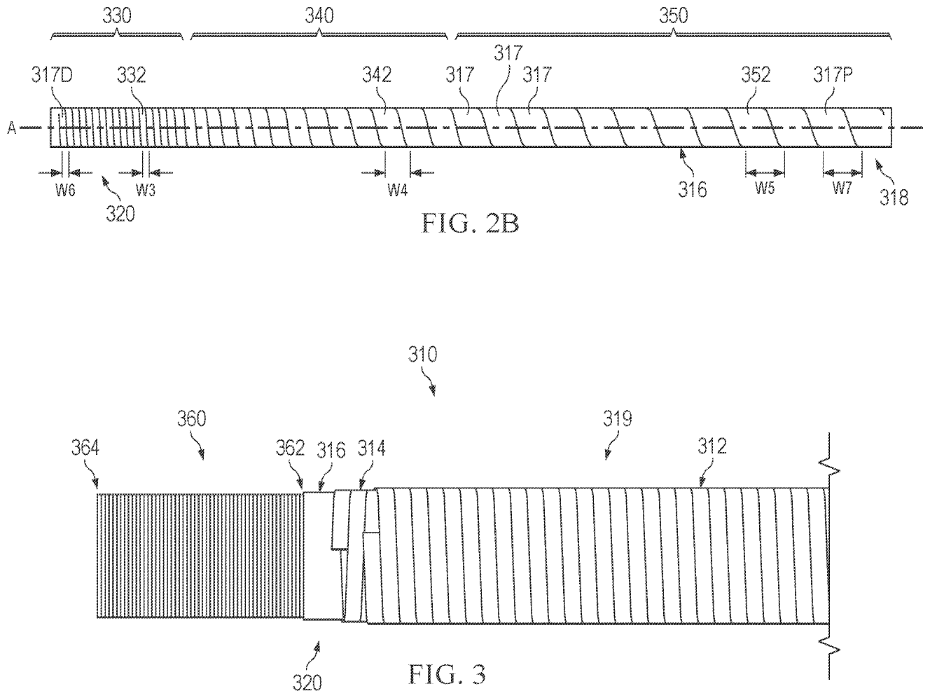

The coil layer 170 may assist in maintaining the patency of a lumen (e.g., the lumen 155 and any other lumens) during articulation or navigation of the elongate device 150. The coil layer 170 may also provide flexibility to the elongate device 150, while enabling a variation in bending stiffness along the length of the elongate device 150. Additionally, the coil layer 170 may provide structural support for the elongate device 150. For example, the coil layer 310 may provide high bi-directional torsional stiffness resistive to twisting or rotational forces and high axial stiffness in both compression and tension. In some examples, the coil layer 170 includes multiple concentric coils, each coil including ribbon elements. The ribbon elements of at least one of the coils may have variable ribbon widths to provide variable bending stiffness for the elongate device 150. The ribbon elements of each coil may have an open pitch or a closed pitch. In other examples, the coil layer 170 includes a woven or braided element. The coil layer 170 may cause the elongate device 150 to have high bi-directional torsional stiffness and high axial stiffness (in both compression and extension). Other examples may include any number or arrangement of support layers and/or coil layers between the outer sheath 160 and the inner sheath 175. In some embodiments, the articulable section(s) of the elongate device 150 may include the coil layer 170. Additional details regarding the coil layer 170 are discussed below in connection with FIGS. 2A-5B.

The inner sheath 175 may include a length of flexible tubing extending from an inner surface to an outer surface, such as to define a certain wall thickness. The inner sheath 175 may be metal, polymer, a composite, or any other suitable material. The inner sheath 175 may include conduits 180 (e.g., four conduits) configured to carry one or more control elements such as pull wires 185. The pull wires 185 may be used to control the articulation of an articulable section of elongate device 150. The pull wires 185 at the articulable section are not surrounded by conduits 188 to allow changes in tension in the pull wires 185 to change the articulation of the articulable section. In some examples, the conduits 180 comprise a cylindrical coil or coil pipe formed, for example, from a narrow ribbon of material. The coiled nature of such a conduit may allow it to bend and twist under tension, compression, and other forces while maintaining the patency of the channel through which the control elements extend. Each conduit 180 may extend within a preformed channel through the inner sheath 175 or may be embedded in the inner sheath 175 as the inner sheath 175 is extruded around the conduits 180. The conduits 180 may be arranged symmetrically about the inner sheath 175. In other examples, the inner sheath 175 may contain any number, type, and arrangement of conduits 180, depending upon the application and structure of the elongate device 150.

The pull wires 185 are disposed coaxially within respective conduits 180. In some examples, the conduits 180 are configured to maintain the patency or openness of the lumen 155 and minimize friction such that the pull wires 185 can slide freely or float within the conduits 180. In some examples, the conduits 180 are configured to maintain the radial spacing or positioning of the pull wires 185 along the length of the elongate device 150.

FIG. 2A illustrates a side view of a coil layer 310 (e.g., the coil layer 170) of an exemplary flexible elongate device (e.g., the flexible elongate device 150). The coil layer 310 provides the elongate device 150 with variable bending stiffness along its length by including one or more coils with ribbon elements having variable ribbon width.

The coil layer 310 may include multiple layers of coils. In some examples, the coil layer 310 may include three layers of coils, such as an outer coil 312, an intermediate coil 314, and an inner coil 316. The outer coil 312, the intermediate coil 314, and the inner coil 316 may be concentric about a longitudinal axis A of the coil layer 310. In some examples, the outer coil 312 defines a lumen, and the intermediate coil 314 may extend within the lumen. The intermediate coil 314 may define a lumen, and the inner coil 316 may extend within the lumen of the intermediate coil 314. In some examples, the coil layer 310 includes more than three coils (e.g., four coils, five coils, etc.). In some examples, the coil layer 310 includes two coils, such as the inner coil 316 and an outer coil. One, some, or all of the outer coil 312, the intermediate coil 314, and the inner coil 316 may have a circular cross-section, a hexagonal cross-section, an octagonal cross-section, or a cross-section of some other shape. Each of the coils of the coil layer 310 provides kink resistance and coil for its kink resistance and structural support.

The coil layer 310 further includes a proximal portion 318 and a distal portion 320. The proximal portion 318 may be adjacent to or closer to a drive unit 204 of a medical instrument system 200 (see FIG. 13). The distal portion 320 is distal to the proximal portion 318 and may be at or adjacent to a distal end 218 of an elongate device 202 of the medical instrument system or a distal end 221 of a non-articulable section 205 of the elongate device 202 (see FIG. 13).

The different coils of the coil layer 310 may include ribbon elements of different ribbon widths. The outer coil 312 includes ribbon elements 313. The intermediate coil 314 includes ribbon elements 315. The inner coil 316 includes ribbon elements 317. In some examples, the ribbon elements 313 of the outer coil 312 have a ribbon width W1. Alternatively, the ribbon elements 313 may have different ribbon widths along the length of the outer coil 312. The ribbon width of the ribbon elements 313 may vary along the length of the outer coil 312 in any arrangement or pattern. In some examples, the ribbon elements 315 of the intermediate coil 314 have a ribbon width W2. Alternatively, the ribbon elements 315 may have different ribbon widths along the length of the intermediate coil 314. The ribbon width of the ribbon elements 315 may vary along the length of the intermediate coil 314 in any arrangement or pattern. In some examples, the ribbon width W2 of the ribbon elements 315 is the same width as the ribbon width W1 of the ribbon elements 313. In some examples, the ribbon elements 317 of the inner coil 316 have ribbon widths that may vary along the length of the inner coil 316, as shown in greater detail in FIG. 2B. In some examples, at least one of the coils of the coil layer 310 (e.g., a coil that does not have variable ribbon width) may be a braided coil formed of at least two woven strands. A braided coil provides high torsional stiffness to the coil layer 310 and therefore to the elongate device 150.

In the example of FIG. 2A, the ribbon elements 317 of the inner coil 316 have variable ribbon width along the length of the coil layer 310. However, in alternative examples the ribbon elements 313 of the outer coil 312 and/or the ribbon elements 315 of the intermediate coil 314 may additionally or alternatively have variable ribbon width along the length of the coil layer 310. In such alternative examples, the ribbon elements 317 of the inner coil 316 may have variable or constant ribbon width. In some examples, the outer coil 312 has a greater influence on the bending stiffness of the coil layer 310 than the intermediate coil 314 and the inner coil 316. In examples when the ribbon elements 313 of the outer coil 312 have variable ribbon width, the coil layer 310 may have a greater range of possible bending stiffness values than when the ribbon elements 315 of the intermediate coil 314 have variable ribbon width or when the ribbon elements 317 of the inner coil 316 have variable ribbon width. In some examples, when the ribbon elements 317 of the inner coil 316 have a constant ribbon width, an interior surface of the inner coil 316 may be smoother than when the ribbon elements 317 have a variable ribbon width. A smooth interior surface of the inner coil 316 may provide a smooth pathway for tools, fluid, or other items to pass through when extending through the lumen of the inner coil 316. Ribbon elements of constant width are most likely manufactured from flat wire and coiled into the required spiraled diameter and pitch necessary for the medical device. Ribbon elements of variable pitch are most likely manufactured by laser cutting a spiral of variable ribbon width into a cylindrical tube. However, both types of ribbon elements could be produced with either manufacturing process or an equivalent process.

FIG. 2B illustrates a side view of the inner coil 316 of the coil layer 310. The ribbon width of the ribbon elements 317 of the inner coil 316 may vary along the length of the coil layer 310 to allow the elongate device 150 to have variable bending stiffness. A wider ribbon width at a region of the coil layer 310 corresponds to a higher bending stiffness at that region of the coil layer 310. Similarly, a narrower ribbon width corresponds to a lower bending stiffness. Accordingly, the ribbon widths of the ribbon elements 317 may be sized to achieve different bending stiffness profiles along the axial length of the elongate device 150.

In some examples, the inner coil 316 may include different regions 330, 340, and 350 along its axial length that have different bending stiffness. The region 330 is a most distal region. The region 340 is proximal of the region 330 and distal of the region 350. The region 350 is a most proximal region. The arrangement and number of regions shown in FIG. 2B is one example. The inner coil 316 may include any other arrangement and/or number of regions.

The ribbon elements 317 of the inner coil 316 may include different ribbon widths to provide for different bending stiffness in the different regions 330, 340, and 350. In some examples, at least one ribbon element 332 in the region 330 has a ribbon width W3. At least one ribbon element 342 in the region 340 has a ribbon width W4. At least one ribbon element 352 in the region 350 has a ribbon width W5. The ribbon width W5 is wider than the ribbon width W4, which is wider than the ribbon width W3. In a region with wide ribbon widths, such as the region 350, the elongate device 150 has a high bending stiffness as compared to regions with narrower ribbon widths. As the ribbon width of the ribbon elements 317 narrows, the bending stiffness of the coil layer 310 decreases, and the corresponding region of the elongate device 150 may become more flexible. Thus, the bending stiffness of the elongate device 150 in the region 350 is the highest, and the bending stiffness of the elongate device 150 in the region 330 is the lowest. The bending stiffness of the elongate device 150 in the region 340 is higher than the bending stiffness in the region 330 but lower than the bending stiffness in the region 350. In the example shown in FIG. 2B, the bending stiffness of the elongate device 150 progressively decreases from a maximum bending stiffness in the region 350 at the proximal portion 318 of the coil layer 310 to a minimum bending stiffness in the region 330 at the distal portion 320 of the coil layer 310. In this manner, the proximal portion 318 is stiffer than the distal portion 320. In some examples, the bending stiffness of the elongate device 150 progressively decreases in a uniform pattern from a maximum bending stiffness in the region 350 at the proximal portion 318 of the coil layer 310 to a minimum bending stiffness in the region 330 at the distal portion 320 of the coil layer 310. In other examples, the bending stiffness of the elongate device 150 progressively decreases in a non-uniform pattern from a maximum bending stiffness in the region 350 at the proximal portion 318 of the coil layer 310 to a minimum bending stiffness in the region 330 at the distal portion 320 of the coil layer 310.

In some examples, the ribbon width of the ribbon elements 317 varies from a narrowest ribbon width W6 to a widest ribbon width W7. For example, the ribbon width of the ribbon elements 317 may progressively increase in a uniform pattern from the narrowest ribbon width W6 to the widest ribbon width W7. Alternatively, the ribbon width of the ribbon elements 317 may progressively increase in a non-uniform pattern from the narrowest ribbon width W6 to the widest ribbon width W7. A more distal ribbon element 317D may have the ribbon width W6, and a more proximal ribbon element 317P may have the widest ribbon width W7. In some examples, when moving from the more distal ribbon element 317D to the more proximal ribbon element 317P, each successive ribbon element may have a wider ribbon width than its preceding ribbon element. Thus, the ribbon width of the ribbon elements 317 may progressively widen from the narrowest ribbon width W6 to the widest ribbon width W7. This may result in a gradient of increasing bending stiffness from the distal portion 320 to the proximal portion 318. In another example, the direction of the bending stiffness gradient may be reversed using successively narrower ribbon widths from the distal portion 320 to the proximal portion 318. In another example, the ribbon widths in each of the regions 330, 340, and 350 may be the same, with ribbon widths being different for the different regions 330, 340, and 350 to achieve different bending stiffness in the regions 330, 340, and 350.

The ribbon widths of the ribbon elements 317 shown in FIG. 2B may be customized, adjusted, or varied in any manner so that the ribbon widths of the ribbon elements 317 are sized in any desired pattern or arrangement to achieve a desired bending stiffness profile along the length of the elongate device 150. For example, the ribbon widths of the ribbon elements 317 may be sized so that the bending stiffness of the elongate device 150 increases from a minimum bending stiffness in the region 350 at the proximal portion 318 of the coil layer 310 to a maximum bending stiffness in the region 330 at the distal portion 320 of the coil layer 310. In this example, the distal portion 320 is stiffer than the proximal portion 318. In another example, the ribbon widths of the ribbon elements 317 may be sized so that the bending stiffness of the elongate device 150 is at a maximum bending stiffness at the proximal and distal portions 318, 320 and is at a minimum bending stiffness at a center point along the axial length of the coil layer 310. In this example, the proximal and distal portions 318, 320 are stiffer than the middle of the elongate device 150. In another example, the ribbon widths of the ribbon elements 317 may be sized so that the bending stiffness of the elongate device 150 remains the same along the axial length of the coil layer 310. The ribbon widths of the ribbon elements 317 may be sized in any other customized configuration to provide greater or less stiffness at selected sections along the length of the coil layer 310.

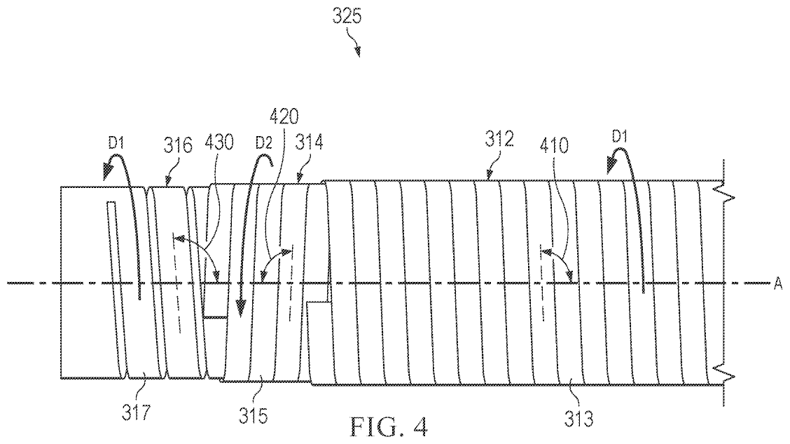

In some examples, the elongate device 150 may include an articulable distal section 360 (e.g., the distal end 218 in FIG. 13), as shown in FIG. 3. A proximal end 362 of the articulable distal section 360 may be coupled to the distal portion 320 of the coil layer 310. In some examples, the coil layer 310 is located in a non-articulable proximal section 319 (e.g., the non-articulable section 205 in FIG. 13) of the elongate device 150 that is proximal to the proximal end 362 of the articulable distal section 360. Conduits 180 (not shown in FIG. 3) that surround the pull wires 185 (not shown in FIG. 3) used to control articulation of the articulable distal section 360 and may extend within the non-articulable proximal section 319, terminating at the articulable distal section 360 or within the articulable distal section 360. For example, the conduits may extend through the non-articulable proximal section 319 within a lumen formed by the coil layer 310, as shown in FIG. 1A. In some examples, the conduits 180 may be between adjacent coils of the coil layer 310. Pull wires 185 may extend through the conduits 180 and extend outside of the conduits 180 to couple to various locations of the articulable distal section 360. The pull wires 185 may be actuated to bend the articulable distal section 360. Bending the articulable distal section 360 may direct a distal end 364 of the articulable distal section 360 in any desired direction, such as along pitch and yaw axes. The elongate device 150 may be steered by bending the articulable distal section 360 and inserting the elongate device 150 within an anatomy (e.g., respiratory passageways). Articulable and steerable elongate devices are described in greater detail in U.S. patent application Ser. No. 16/877,760, filed on May 19, 2020, titled “Flexible Instrument with Grooved Steerable Tube,” which is incorporated by reference herein in its entirety. FIG. 3 shows the outer coil 312 and the intermediate coil 314 ending proximal of the distal portion 320 of the coil layer 310. In some examples, the outer coil 312 and/or the intermediate coil 314 continue along at least a portion of the axial length to the articulable distal section 360.

The outer coil 312, the intermediate coil 314, and the inner coil 316 of the coil layer 310 may have different configurations, such as different pitch directions and/or different sizes or shapes. A pitch direction refers to a direction of alignment of the ribbon elements of a coil, such as may be defined by the winding direction of a coil in the example where the ribbon elements are formed from a wire or by the direction of slits between ribbon elements in the example where the coil is formed from a hypotube. Adjacent coils of the coil layer 310 may have opposing pitch directions to accommodate tight bending (e.g., a small radius of curvature). For example, when the outer coil 312 and intermediate coil 314 have opposing pitch directions, the outer coil 312 and intermediate coil 314 may be able to bend to a tighter radius of curvature than a single coil is able to bend or than two coils with the same pitch direction are able to bend without causing the material of the outer coil 312 and intermediate coil 314 to be permanently deformed. Having the outer coil 312 and intermediate coil 314 include opposing pitch directions may also help prevent one or more ribbon elements 315 of the intermediate coil 314 from being stuck in a gap 311 between two or more ribbon elements 313 of the outer coil 312 when the elongate device 150 is bent.

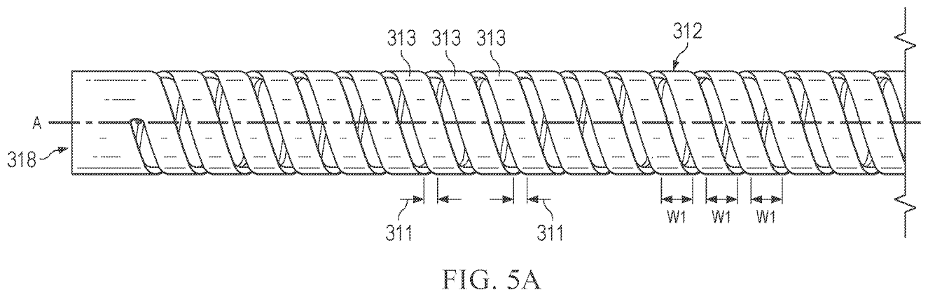

For example, FIG. 4 illustrates a side view of a portion 325 of the coil layer 310. The portion 325 may be positioned at any axial location along the length of the coil layer 310 (e.g., at the distal portion 320, near the distal portion 320, at the proximal portion 318, near the proximal portion 318, or anywhere between the distal portion 320 and the proximal portion 318). The ribbon elements 313 of the outer coil 312 are oriented in a pitch direction D1, the ribbon elements 315 of the intermediate coil 314 are oriented in a pitch direction D2, and the ribbon elements 317 of the inner coil 316 are oriented in the pitch direction D1. In examples when the outer coil 312 is a wire, the pitch direction D1 may indicate a winding direction of the ribbon elements 313 of the outer coil 312. In examples when the outer coil 312 is a hypotube, the pitch direction D1 may indicate a spiral direction of the helical ribbon elements 313 of the outer coil 312. In examples when the intermediate coil 314 is manufactured from a wire, the pitch direction D2 may indicate a winding direction of the ribbon elements 315 during the winding or coiling process. In examples when the intermediate coil 314 is cut from a hypotube, the pitch direction D2 may indicate a spiral direction of the cut pattern of the intermediate coil 314. In examples when the inner coil 316 is manufactured from or coiled from a wire, the pitch direction D1 may indicate a winding direction of the ribbon elements 317 of the inner coil 316. In examples when the inner coil 316 is a hypotube, the pitch direction D1 may indicate a spiral direction of the cut pattern to create the helical ribbon elements 317 of the inner coil 316.

At least one coil of the coil layer 310 may be oriented in a pitch direction that is different from the pitch direction of the other coils of the coil layer 310. For example, as shown in FIG. 4, the outer coil 312 and the inner coil 316 are both oriented in the pitch direction D1, and the intermediate coil 314 is oriented in the pitch direction D2. In other examples, the outer coil 312 may be oriented in a different pitch direction (e.g., the pitch direction D1) than the intermediate coil 314 and the inner coil 316, which may both be oriented in the pitch direction D2. In other examples, the inner coil 316 may be oriented in a different pitch direction (e.g., the pitch direction D1) than the outer coil 312 and the intermediate coil 314, which may both be oriented in the pitch direction D2. The outer coil 312, the intermediate coil 314, and the inner coil 316 may be oriented in any other pitch direction combination where one of outer coil 312, the intermediate coil 314, or the inner coil 316 is oriented in a pitch direction that is different from the pitch direction of the other coils. The outer coil 312 may be oriented in the pitch direction D1 or the pitch direction D2. The intermediate coil 314 may be oriented in the pitch direction D1 or the pitch direction D2. The inner coil 316 may be oriented in the pitch direction D1 or the pitch direction D2.

FIG. 4 shows pitch angles 410, 420, 430 relative to the longitudinal axis A of the coil layer 310. The pitch angle 410 is a pitch angle of the ribbon elements 313 of the outer coil 312. The pitch angle 420 is a pitch angle of the ribbon elements 315 of the intermediate coil 314. The pitch angle 430 is a pitch angle of the ribbon elements 317 of the inner coil 316. The pitch angle between the coils of the coil layer 310 (e.g., the outer coil 312, the intermediate coil 314, and the inner coil 316) may affect the ability of the coil layer 310 to resist elongation of the coil layer 310 (e.g., over-stretching of the coil layer 310 that results in axial deformation of the coil layer 310). For example, when the pitch angles of the coils of the coil layer 310 are within a close range (e.g., a 10° range, a 15° range, or a 20° range) the coil layer 310 may have a higher resistance to elongation than when the pitch angles are within a wide range (e.g., a 45° range, a 60° range, or a 90° range). As the pitch angles of the ribbon elements of the coils of the coil layer 310 approach 0° or 180°, resistance to elongation approaches a maximum resistance to elongation. As the pitch angles of the ribbon elements of the coils of the coil layer 310 approach 90°, resistance to elongation approaches a minimum resistance to elongation. In some examples, if the pitch angle 410 is at 120°, the pitch angle 420 is at 80°, and the pitch angle 430 is at 120°, the coil layer 310 may have a higher resistance to elongation than when the pitch angles are closer to 90°. In some examples, if the pitch angle 410 is at 95°, the pitch angle 420 is at 85°, and the pitch angle 430 is at 150°, the coil layer 310 may have a higher resistance to elongation than when all three of the pitch angles are closer to 90°. Additionally, in some examples, the pitch angle 430 of the ribbon elements 317 of the inner coil 316 may be close to 90°(e.g., 85°, 87°, 92°, or 95°), and the pitch angle 420 of the ribbon elements 315 of the intermediate coil 314 and/or the pitch angle 410 of the ribbon elements 313 of the outer coil 312 may be further from 90° (e.g., 60°, 70°, 110°, or 120°). In such examples, the coil layer 310 may provide a smooth passage for a tool extending through the lumen of the inner coil 316 while maintaining resistance to elongation. In the example shown in FIG. 4, the coil layer 310 may have a higher resistance to elongation than a coil layer with coils that have pitch angles within a wide range. For example, the pitch angle 410 may be 95°, the pitch angle 420 may be 85°, and the pitch angle 430 may be 100°. The pitch angle 410, the pitch angle 420, and the pitch angle 430 are within a 15°range of each other, which may be a close range. One or more of the pitch angle 410, the pitch angle 420, and the pitch angle 430 may be adjusted to adjust the resistance to elongation of the coil layer 310.

In some examples, if the pitch angle 410 is at 60°, the pitch angle 420 is at 80°, and the pitch angle 430 is at 60°, the coil layer 310 may have a higher resistance to elongation than when the pitch angles are closer to 90°. In some examples, if the pitch angle 410 is at 85°, the pitch angle 420 is at 85°, and the pitch angle 430 is at 75°, the coil layer 310 may have a higher resistance to elongation than when all three of the pitch angles are closer to 90°. Additionally, in some examples, the pitch angle 430 of the ribbon elements 317 of the inner coil 316 may be close to 90° (e.g., 85°, 86°, 87°, 88°, or 89°), and the pitch angle 420 of the ribbon elements 315 of the intermediate coil 314 and/or the pitch angle 410 of the ribbon elements 313 of the outer coil 312 may be further from 90° (e.g., 50°, 60°, or 70°). In such examples, the coil layer 310 may provide a smooth passage for a tool extending through the lumen of the inner coil 316 while maintaining resistance to elongation. In the example shown in FIG. 4, the coil layer 310 may have a higher resistance to elongation than a coil layer with coils that have pitch angles within a wide range. For example, the pitch angle 410 may be 85°, the pitch angle 420 may be 85°, and the pitch angle 430 may be 80°. The pitch angle 410, the pitch angle 420, and the pitch angle 430 are within a 15° range of each other, which may be a close range. One or more of the pitch angle 410, the pitch angle 420, and the pitch angle 430 may be adjusted to adjust the resistance to elongation of the coil layer 310.

In some examples, the pitch angle 410, the pitch angle 420, and the pitch angle 430 are different angles. In some examples, two or more of the pitch angle 410, the pitch angle 420, and the pitch angle 430 may be the same. In some examples, the coils that are oriented in the same pitch direction may have the same pitch angle. For example, if the outer coil 312 and the inner coil 316 are oriented in the pitch direction D1, the pitch angle 410 of the outer coil 312 and the pitch angle 430 of the inner coil 316 may be the same angle.

When adjacent coils are oriented in opposing pitch directions (or when at least two coils are oriented in opposing pitch directions), the elongate device 150 may be better able to accommodate tight bending (e.g., a small radius of curvature). For example, a single coil or multiple coils oriented in the same pitch direction may result in the elongate device 150 being susceptible to permanent kinking and/or deforming during bending of the elongate device 150. In examples when two coils of the coil layer 310 are oriented in an opposing pitch direction, the coil layer 310 may provide increased stiffness to the elongate flexible device 150 such that the elongate flexible device 150 can bend to a small radius of curvature without permanent kinking or deforming. Additionally, when adjacent coils are oriented in opposing pitch directions (or when at least two coils are oriented in opposing pitch directions), the elongate device 150 may have greater torsional resistance than when adjacent coils are oriented in the same pitch directions. In some examples, when the coil layer 310 includes three coils (e.g., the outer coil 312, the intermediate coil 314, and the inner coil 316) that are oriented in alternating pitch directions, the elongate device 150 has bi-directional torsional resistance. For example, when torque is applied in one direction, the inner coil 316 may expand, the intermediate coil 314 may contract, and the outer coil 312 may expand. When torque is applied in the opposite direction, the inner coil 316 may contract, the intermediate coil 314 may expand, and the outer coil 312 may contract. The resulting expansion and contraction between the coils when torque is applied in both directions results in the elongate device 150 having bi-directional torsional resistance. When adjacent coils of the coil layer 310 have opposite pitch directions, the likelihood of ribbon elements of a coil being stuck in gaps formed between ribbon elements of an adjacent coil when the coil layer 310 is bent is reduced. For example, the ribbon elements 315 of the intermediate coil 314 are less likely to be stuck in a gap between two or more of the ribbon elements 313 of the outer coil 312 or between two or more of the ribbon elements 317 of the inner coil 316 when the elongate device 150 is bent.

In some examples, one, some, or all of the outer coil 312, the intermediate coil 314, and inner coil 316 may be closed pitch. When a coil is closed pitch, no gap extends between the ribbon elements of the coil when the coil layer 310 is unbent. In some examples, one, some, or all of the outer coil 312, the intermediate coil 314, and inner coil 316 may be open pitch. When a coil is open pitch, a gap extends between the ribbon elements of the coil.

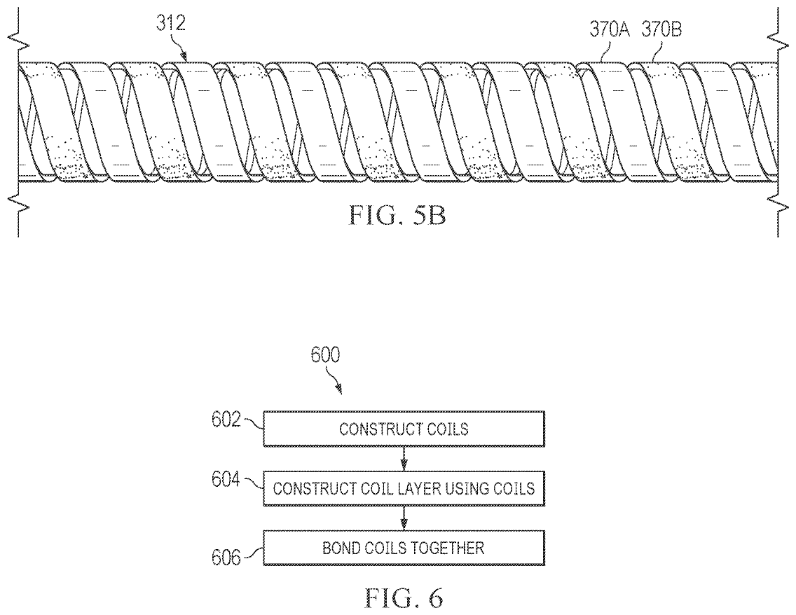

FIGS. 5A and 5B illustrate a side view of the outer coil 312 having an open pitch. An open pitch refers to the coil having a gap between adjacent ribbon elements when the coil is unbent. One or more coils of a coil layer 310 may have an open pitch. While FIGS. 5A and 5B discuss the outer coil 312, the discussion applies to each of the intermediate coil 314 and the inner coil 316. In examples when one or more coils of the coil layer 310 have an open pitch, the coil(s) includes a gap between one or more ribbon elements of the coil(s). For example, the outer coil 312 may include a gap 311 between one or more ribbon elements 313 of the outer coil 312. In some examples, each of the gaps 311 between the ribbon elements 313 is the same width. Alternatively, one or more of the gaps 311 may be different widths. The widths of the gaps 311 between the ribbon elements 313 may vary along the length of the outer coil 312 in any arrangement or pattern.

In some examples, a coil may include multiple ribbons that are interwoven. As shown in FIG. 5B, the outer coil 312 includes two ribbons 370A, 370B that are interwoven. In some examples, the intermediate coil 314 and/or the inner coil 316 additionally or alternatively include multiple ribbons.

In some examples, one or more coils of the coil layer 310 may be manufactured or coiled from wires. The coil may be made or formed with multi-filar wires or single filar wires. In FIG. 5A, for example, the outer coil 312 is a single filar wire. The intermediate coil 314 and/or the inner coil 316 may also be single filar wires. In some examples, one or more of the outer coil 312, the intermediate coil 314, and the inner coil 316 may be multi-filar wires (see the outer coil 312 in FIG. 5B, for example, where each of the ribbons 370A and 370B are made from one wire). In some examples, the ribbon(s) of one or more of the outer coil 312, the intermediate coil 314, or the inner coil 316 may be formed from a round wire. In some examples, the ribbon(s) of one or more of the outer coil 312, the intermediate coil 314, or the inner coil 316 may be formed from a flat wire. Any combination of single filar wires, multi-filar wires, round wires, flat wires, open pitch coils, closed pitch coils, variable ribbon width, and consistent ribbon width may be used in the coil layer 310.

In some examples, the ribbon(s) of one or more of the outer coil 312, the intermediate coil 314, or the inner coil 316 may be formed from a tube, such as a hypotube. The tube may be laser cut to create slits that define the ribbon elements, pitch direction, and pitch angle of the coil. In some examples, the tube may be water jet cut to create the ribbon elements from the tube. In other examples, the tube may be plasma cut or mechanically cut to create the ribbon elements from the tube. In some examples, one or more of the outer coil 312, the intermediate coil 314, or the inner coil 316 may be 3D printed. For example, the coil with the variable ribbon width, such as the inner coil 316, may be 3D printed. In some examples, the coil with the variable ribbon width is made from a wire. The wire may have variable cross-sectional sizes along its length to provide the variable ribbon widths, and the coil may be created by winding the wire to create ribbon elements of different ribbon width.

Each coil of the coil layer 310 may be made of metal, polymer, a composite, or any other suitable material. For example, one or more of the outer coil 312, the intermediate coil 314, and the inner coil 316 may be made of stainless steel. In another example, one or more of the outer coil 312, the intermediate coil 314, and the inner coil 316 may be made from a carbon fiber epoxy composite. All of the coils of the coil layer 310 may be made from the same materials, or different coils may be made from different materials.

In some examples, adjacent coils of the coil layer 310 may be bonded or otherwise fixed together at various locations along the length of the coil layer 310. For example, the outer coil 312, the intermediate coil 314, and the inner coil 316 may be bonded together at the proximal portion 318 and the distal portion 320 of the coil layer 310. In other examples, adjacent coils are not bonded to each other.

The bonding technique used to bond adjacent coils may be any type of appropriate bonding technique that is suitable to the specific materials used for the coils. For example, adjacent metal coils may be welded together at one or more welding locations. The type of weld(s) used to weld adjacent coils together may be any one or more of a spot weld, an electroresistive weld, a seam weld, a laser weld, any combination thereof, and/or any other type of weld. Additionally or alternatively, adjacent coils may be bonded together with an adhesive or other bonding material.

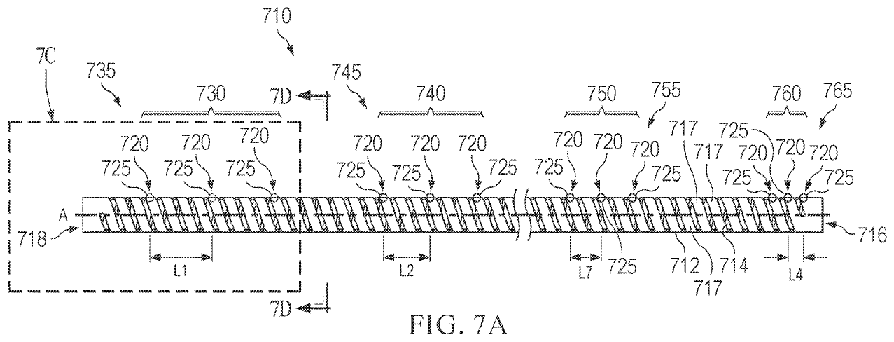

FIG. 6 is a flowchart illustrating a method 600 of manufacturing a coil layer (e.g., the coil layer 310) of a flexible elongate device (e.g., the flexible elongate device 150). The method 600 will be discussed with reference to FIGS. 2A and 2B. The method 600 is illustrated as a set of operations or processes 602 through 606. The processes illustrated in FIG. 6 may be performed in a different order than the order shown in FIG. 6, and one or more of the illustrated processes might not be performed in some examples of the method 600. Additionally, one or more processes that are not expressly illustrated in FIG. 6 may be included before, after, in between, or as part of the illustrated processes.

At a process 602, at least two coils are constructed. For example, at least two of the outer coil 112, the intermediate coil 114, and the inner coil 116 are constructed. For example, one or more of the coils may be tubes, such as hypotubes, which may be laser cut to create slits or gaps that define ribbon elements, pitch direction, and pitch angle of the coils. In some examples, the tubes may be water jet cut to create the ribbon elements from the tube. In other examples, the tubes may be plasma cut or mechanically cut to create the ribbon elements from the tube. In some examples, one or more of the coils may be 3D printed. In some examples, one or more of the coils may be formed or wound from a wire. The coil(s) may be created by winding the wire to create ribbon elements of different ribbon width or different pitch.

At a process 604, the coil layer 310 may be constructed using at least two of the outer coil 112, the intermediate coil 114, and the inner coil 116. For example, the inner coil 116 may be positioned within a lumen of the intermediate coil 114, and/or the intermediate coil 114 may be positioned within a lumen of the outer coil 112.

At a process 606, the coils of the coil layer 310 may be bonded together. For example, the coils of the coil layer 310 may be welded together and/or bonded together with an adhesive or other bonding material. The coils may be bonded together at one or more locations along the length of the coils.

In examples when the coils are welded together, the location and number of welds will change the bending stiffness of the coil and hence the bending stiffness of the medical device. For example, welds on consecutive ribbons will increase the bending stiffness whereas welds on every fifth ribbon would have less impact on the bending stiffness. Alternatively, several welds between inner and outer coils around the circumference of the coil increases the bending stiffness more than just a single weld in one location on the circumference. A variable stiffness shaft may be constructed by welding every ribbon in the proximal section of the medical device, welding every firth ribbon in the midsection, and welding every tenth ribbon in the distal section. Alternative bending stiffness profiles may be achieved by alternative welding profiles, as discussed in more detail below. For example, the outer coil 312 and the intermediate coil 314 may be welded together. Additionally or alternatively, the intermediate coil 314 and the inner coil 316 may be welded together. Additionally or alternatively, the outer coil 312, the intermediate coil 314, and the inner coil 316 may all be welded together.

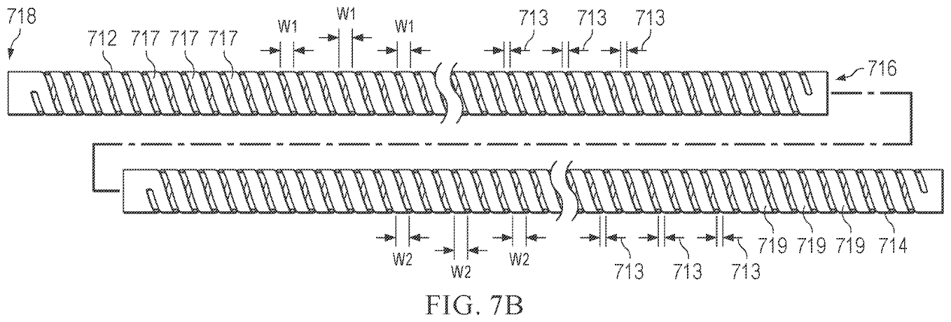

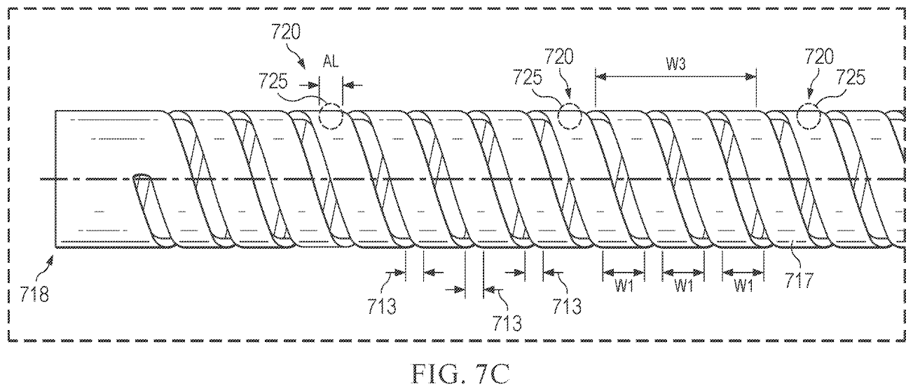

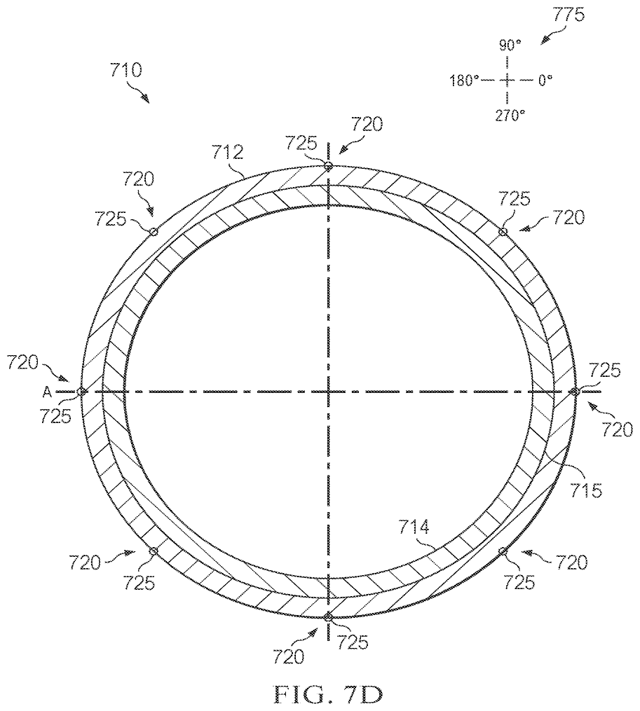

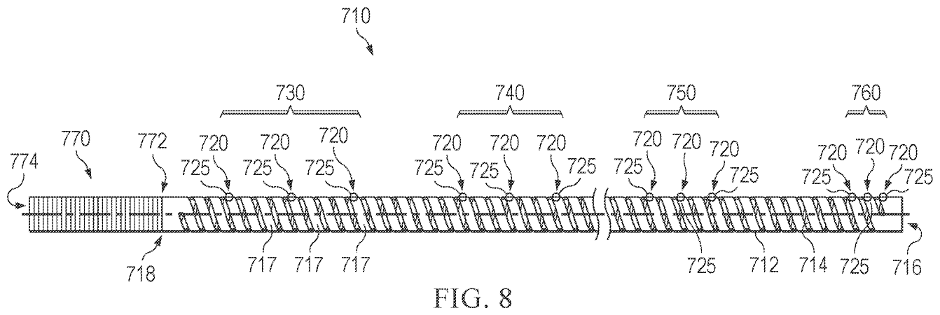

FIG. 7A illustrates a side view of a coil layer 710 (e.g., the coil layer 310 and/or the coil layer 170) of an exemplary flexible elongate device (e.g., the flexible elongate device 150). The elongate device 150 may be a catheter, an instrument (e.g., a medical instrument), a sheath, or any other flexible elongate body. The coil layer 710 may assist in maintaining the patency of a lumen (e.g., the lumen 155) of the elongate device 150 (and any other lumens) during articulation of the elongate device 150. The coil layer 710 may also provide flexibility to the elongate device 150, while allowing for variable bending stiffness of the elongate device 150. The coil layer 710 may also provide structural support for the elongate device 150. The elongate device 150 may also have components other than the coil layer 710 which are not shown in FIG. 2A, such as the components discussed above with respect to FIGS. 1A and 1B. In some examples, the elongate device 150 has variable bending stiffness along its length.

The coil layer 710 may include at least two layers of coils, such as an outer coil 712 (e.g., the outer coil 312) and an inner coil 714 (e.g., the intermediate coil 314 or the inner coil 316). FIG. 7B illustrates a side view of the outer coil 712 and the inner coil 714. In some examples, the outer coil 712 defines a lumen, and the inner coil 714 may extend within the lumen. In some examples, the coil layer 710 includes more than two coils (e.g., three coils, four coils, etc.). The coil layer 710 further includes a proximal portion 716 (e.g., the proximal portion 318) and a distal portion 718 (e.g., the distal portion 320). The proximal portion 716 may be adjacent to a drive unit 204 of a medical instrument system 200 (see FIG. 13). The distal portion 718 is distal to the proximal portion 716 and may be at or adjacent to a distal end 218 of an elongate device 202 of the medical instrument system (see FIG. 13). The outer coil 712 includes ribbon elements 717. The inner coil 714 includes ribbon elements 719. In some examples, each of the ribbon elements 717 of the outer coil 712 has a ribbon width W1. Alternatively, one or more of the ribbon elements 717 may have different ribbon widths, as discussed above with respect to the coil layer 310. The ribbon widths of the ribbon elements 717 may vary along the length of the outer coil 712 in any arrangement or pattern, as discussed above with respect to the coil layer 310. In some examples, each of the ribbon elements 719 of the inner coil 714 has a ribbon width W2. Alternatively, one or more of the ribbon elements 719 may have different ribbon widths, as discussed above with respect to the coil layer 310. The ribbon widths of the ribbon elements 719 may vary along the length of the inner coil 714 in any arrangement or pattern, as discussed above with respect to the coil layer 310. In some examples, the ribbon width W2 of one or more of the ribbon elements 719 is the same width as the ribbon width W1 of one or more of the ribbon elements 717.

In some examples, the outer coil 712 may include a gap 713 between one or more ribbon elements 717 of the outer coil 712. The inner coil 714 may include a gap 713 between one or more ribbon elements 719 of the inner coil 714. In some examples, each of the gaps 713 between the ribbon elements 717 of the outer coil 712 has the same width. Alternatively, one or more of the gaps 713 between the ribbon elements 717 may have different widths. The widths of the gaps 713 between the ribbon elements 717 may vary along the length of the outer coil 712 in any arrangement or pattern. In some examples, each of the gaps 713 between the ribbon elements 719 of the inner coil 714 has the same width. Alternatively, one or more of the gaps 713 between the ribbon elements 719 may have different widths. The widths of the gaps 713 between the ribbon elements 719 may vary along the length of the inner coil 714 in any arrangement or pattern. In some examples, the width of one or more of the gaps 713 between the ribbon elements 719 is the same width as the width of one or more of the gaps 713 between the ribbon elements 717.

FIGS. 7A and 7B show the ribbon elements 717 of the outer coil 712 and the ribbon elements 719 of the inner coil 714 ending proximal of the distal portion 718 of the coil layer 710. In some examples, the ribbon elements 717 of the outer coil 712 and the ribbon elements 719 of the inner coil 714 continue all the way to the distal portion 718. FIGS. 7A and 7B also show the ribbon elements 717 of the outer coil 712 and the ribbon elements 719 of the inner coil 714 ending distal of the proximal portion 716 of the coil layer 710. In some examples, the ribbon elements 717 of the outer coil 712 and the ribbon elements 719 of the inner coil 714 continue all the way to the proximal portion 716. One or both of the outer coil 712 and inner coil 714 may have a circular cross-section, a hexagonal cross-section, an octagonal cross-section, or a cross-section of some other shape.

The outer coil 712 and inner coil 714 may be bonded together with one or more bonds 725 located at one or more bonding locations 720 along an axial length of the coil layer 710 which extends along an axis A. The bonds 725 may be spaced apart from each other at variable distances to allow the elongate device 150 to have variable bending stiffness that may be tuned to a customized bending stiffness profile. A higher spatial density of bonds 725 at a region of the coil layer 710 corresponds to a higher bending stiffness at that region of the coil layer 710. Similarly, a lower spatial density of bonds 725 corresponds to a lower bending stiffness. Accordingly, the bonds 725 may be positioned at different bonding locations 720 to achieve different bending stiffness profiles along the axial length of the elongate device 150.

In some examples, the coil layer 710 may include different regions 730, 740, 750, 760 along its axial length that have different bending stiffness. The region 730 is a most distal region. The region 740 is proximal of the region 730 and distal of the region 750. The region 750 is proximal of the region 740 and distal of the region 760. The region 760 is a most proximal region. The arrangement and number of regions shown in FIG. 7A is one example. Any other arrangement (e.g., order, length) or number of regions may be used.

The regions 730, 740, 750, 760 include a respective subset 735, 745, 755, 765 of bonds located at the bonding locations 720. The bonds 725 in the subset 735 of bonds in the region 730 are axially spaced apart from each other by a length L1. The bonds 725 in the subset 745 of bonds in the region 740 are axially spaced apart from each other by a length L2. The bonds 725 in the subset 755 of bonds in the region 750 are axially spaced apart from each other by a length L3. The bonds 725 in the subset 765 of bonds in the region 760 are axially spaced apart from each other by a length LA. As the length between the bonds 725 decreases, the spatial density of the bonds 725 increases. The length L1 is longer than the length L2, which is longer than the length L3, which is longer than the length LA. Thus, a spatial density of the bonds 725 in the region 760 is higher than a spatial density of the bonds 725 in the region 750, which is higher than a spatial density of the bonds 725 in the region 740, which is higher than a spatial density of the bonds 725 in the region 730.

In a region with a high spatial density of bonds 725, such as the region 760, the elongate device 150 has a high bending stiffness. As the spatial density of the bonds 725 decreases, the bending stiffness of the elongate device 150 decreases, and the elongate device 150 becomes more flexible. Thus, the bending stiffness of the elongate device 150 in the region 760 is the highest, and the bending stiffness of the elongate device 150 in the region 730 is lowest. The bending stiffness of the elongate device 150 in the region 740 is higher than the bending stiffness in the region 730 but lower than the bending stiffness in the region 750. The bending stiffness of the elongate device 150 in the region 750 is higher than the bending stiffness in the region 740 but lower than the bending stiffness in the region 760. In the example shown in FIG. 7A, the bending stiffness of the elongate device 150 variably decreases from a maximum bending stiffness in the region 760 at the proximal portion 716 of the coil layer 710 to a minimum bending stiffness in the region 730 at the distal portion 718 of the coil layer 710. In this manner, the proximal portion 716 is stiffer than the distal portion 718. Each region 730, 740, 750, and 760 is shown as having a consistent spacing between bonds 725, but the spacing between adjacent bonds 725 within a region is not necessarily the same.

The bending stiffness of the elongate device 150 may be tuned to a customized bending stiffness profile by customizing the locations where the outer coil 712 and inner coil 714 are bonded. The bending stiffness may be varied along the axial length of the coil layer 710, for example, when the bonds 725 are selected to be at locations with varied spacing along the axial length. In some examples, more or fewer bonding locations 720 than those shown in FIG. 7A may be used. The bonding locations 720 shown in FIG. 7A may be selected, customized, adjusted, or varied in any manner so that the bonding locations 720 are positioned at any desired positions in any desired pattern or arrangement. For example, the bonding locations 720 may be defined so that the bending stiffness of the elongate device 150 variably increases from a minimum bending stiffness in the region 760 at the proximal portion 716 of the coil layer 710 to a maximum bending stiffness in the region 730 at the distal portion 718 of the coil layer 710. In this example, the distal portion 718 is stiffer than the proximal portion 716.

In another example, the bonding locations 720 may be defined so that the bending stiffness of the elongate device 150 is at a maximum bending stiffness at the proximal and distal portions 716, 718 and is at a minimum bending stiffness at a center point along the axial length of the coil layer 710. In this example, the proximal and distal portions 716, 718 are stiffer than the middle of the elongate device 150. In another example, the bonding locations 720 may be defined so that the bending stiffness of the elongate device 150 remains the same along the axial length of the coil layer 710. The bonding locations 720 may be positioned at any other locations in any other customized configuration to provide greater or less stiffness at selected sections along the length of the coil layer 710.

In some examples, the coil layer 710 includes more than two coils (e.g., three coils, four coils, etc.). In examples when the coil layer 710 includes three coils, the bonds 720 may be between a first and second coil or the bonds 720 may be between the second and third coil. In some examples, some bonds 720 may be between the first and second coil and other bonds 720 may be between the second and third coil. The bonds 720 may have variable spatial density to achieve variable bending stiffness as discussed above.

One or more of the bonds 725 have an axial length AL, as shown in FIG. 7C. In some examples, each of the bonds 725 may have the same axial length AL. In other examples, some or all of the bonds 725 have different axial lengths AL. For example, the axial length AL of one or more of the bonds 725 may be equal to a ribbon width W1 of one ribbon element 717 of the outer coil 712. In some examples, the axial length of one or more of the bonds 725 may span three ribbon elements 717 of the outer coil 712. For example, the axial length AL of one or more of the bonds 725 may be equal to a ribbon width W3 of three ribbon elements 717 of the outer coil 712. The bonds 725 may have any other axial length. A longer axial bond length results in a higher bending stiffness at that particular bonding location. The bending stiffness profile of the elongate device 150 may be further tuned and customized by varying the axial bond length of one or more of the bonds 725 as well as varying the spatial density of the bonds 725 as discussed above.

In some examples, the outer coil 712 may include a gap 713 between one or more ribbon elements 717 of the outer coil 712. The axial length AL of one or more of the bonds 725 may span from one gap 713 to another gap 713, such as when the axial length AL is equal to the ribbon width W1 of the outer coil 712. In some examples, the axial length AL of one or more bonds 725 may be shorter than the ribbon width W1.

Bonds 725 may vary in spatial density along different dimensions to achieve variable bending stiffness for the coil layer 710. While FIG. 7A shows the bonding locations 720 only on one side of the coil layer 710 (e.g., at the top side), multiple bonding locations 720 may be located along a radial length (e.g., along an arc 715 of the coil layer 710 defined between the outer coil 712 and the inner coil 714) of the elongate device 150. For example, as shown in FIG. 7D, the bonds 725 may be located at bonding locations 720 spaced around the arc 715. In some examples, more or fewer bonding locations 720 than those shown in FIG. 7D may be used. The bonding locations 720 may be selected, customized, adjusted, or varied in any manner so that the bonding locations 720 are positioned at any desired positions in any desired pattern or arrangement.

The spatial density of the bonds 725, defined by the space between adjacent bonding locations 720, may vary along the arc 715. Generally, the higher the spatial density of bonds 725 along the arc 715, the higher the bending stiffness of the coil layer 710 at or near the arc 715. For example, bonds 725 may be positioned at one or more of the bonding locations 720 (e.g., at a 0° position, a 90° position, a 180° position, or a 270° position as defined by the axis 775), or any other radial position, along the arc 715. This may allow for the bending stiffness of the elongate device 150 to be varied in one or more bending planes. For example, if all the bonding locations 720 are positioned above the bending plane A-A (e.g., between the 0° and 180° positions), the bending stiffness of the elongate device 150 will be greater above the bending plane A-A than below the bending plane A-A.

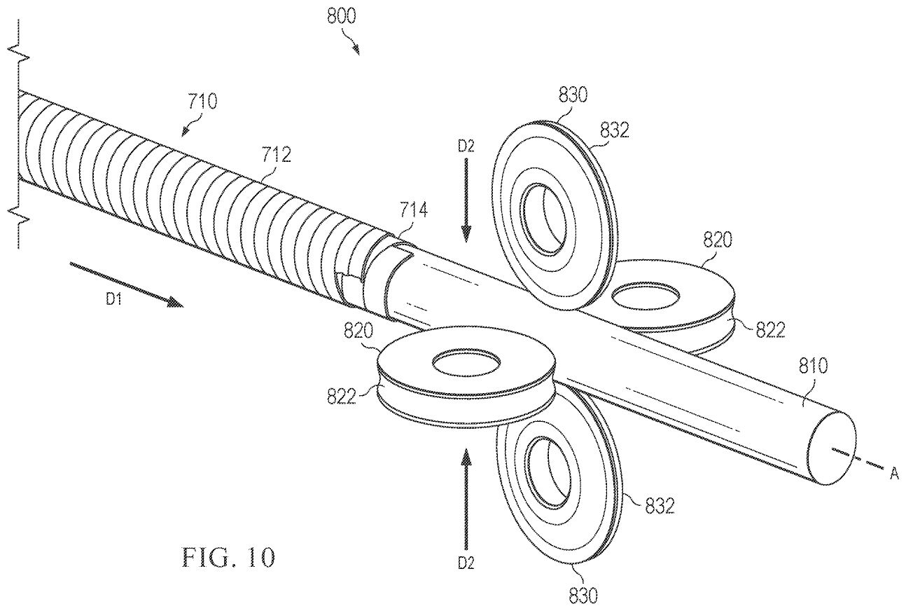

In some examples, the bonding locations 720 may be radially paired at 180° radial intervals, which will be discussed in further detail below with respect to FIG. 10. For example, if one bonding location 720 is at the 90° position, another, paired bonding location 720, may be at the 270° position. Similarly, if one bonding location 720 is at the 0° position, another, paired bonding location 720, may be at the 180° position. The bonding locations 720 may be paired at any other radial interval (e.g., a 30° and 210° pair, a 45° and 225° pair, etc.). The bonding locations 720 may be grouped at any other radial interval that may be more or less than a 180° radial interval. For example, one bonding location at the 45° position may be grouped with another bonding location at the 135° position. In some examples, three or more bonding locations 720 may be grouped together (e.g., bonding locations at the 0°, 90°, and 180° positions).

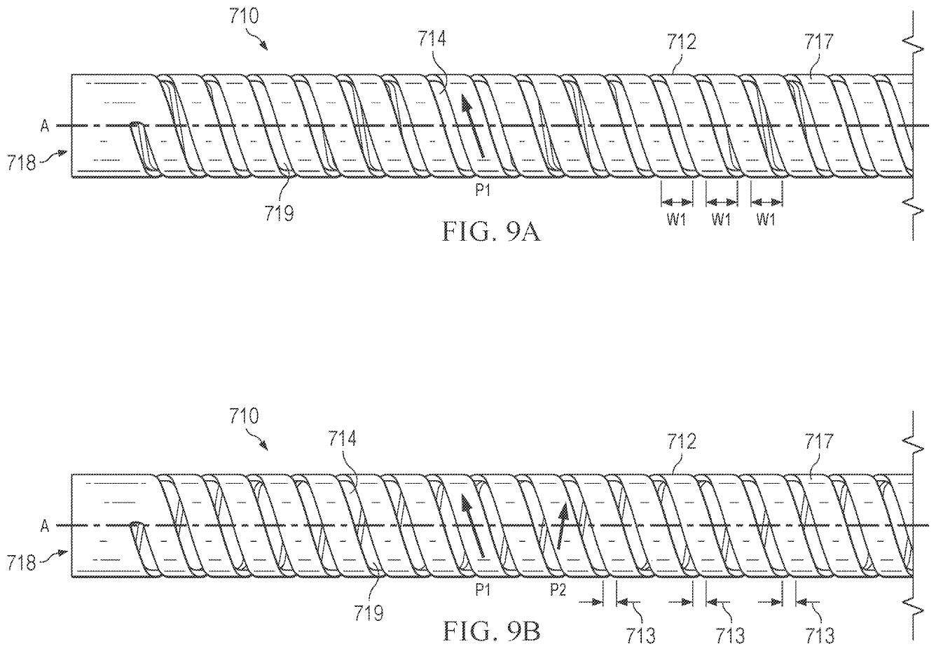

In some examples, the elongate device 150 may include an articulable distal section 770 (e.g., the articulable distal section 370), as shown in FIG. 8, which may be the distal end 218 in FIG. 13. A proximal end 772 of the articulable distal section 770 may be coupled to the distal portion 718 of the coil layer 710. In some examples, the coil layer 710 is located in a non-articulable proximal section of the elongate device 150 that is proximal to the proximal end 772 of the articulable distal section 770. Conduits (not shown) may extend through the non-articulable coil layer 710. The conducts may also extend into some or all of the articulable distal section 770. Pull wires (not shown) may extend through the conduits and may be coupled to various locations of the articulable distal section 770. The pull wires may be actuated to bend the articulable distal section 770. Bending the articulable distal section 770 may direct a distal end 774 of the articulable distal section 770 in any desired direction, such as along pitch and yaw axes. The elongate device 150 may be steered by bending the articulable distal section 770 and inserting the elongate device 150 within an anatomy (e.g., respiratory passageways). Articulable and steerable elongate devices are described in detail in U.S. patent application Ser. No. 16/877,760, filed on May 19, 2020, titled “Flexible Instrument with Grooved Steerable Tube,” which is incorporated by reference herein in its entirety. FIG. 8 shows the ribbon elements 717 of the outer coil 712 and the ribbon elements 719 of the inner coil 714 ending proximal of the distal portion 718 of the coil layer 710. In some examples, the ribbon elements of the outer coil 712 and inner coil 714 continue all the way up to the articulable distal section 770.

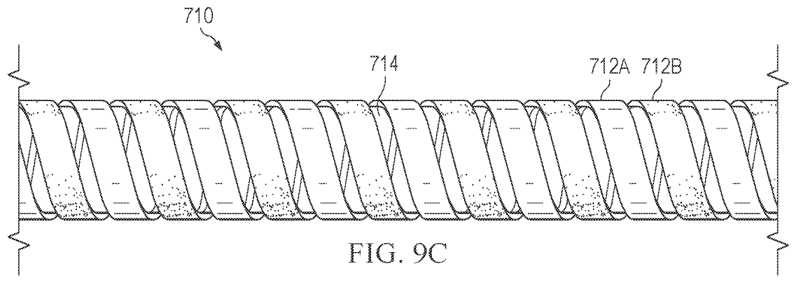

The outer coil 712 and inner coil 714 of the coil layer 710 may be wound in various configurations and/or may be sized and shaped in various different configurations. For example, FIG. 9A illustrates a side view of a distal portion of the coil layer 710. The ribbon elements 717 of the outer coil 712 and the ribbon elements 719 of the inner coil 714 have the same pitch direction P1, which is measured in relation to a longitudinal axis A of the coil layer 710. In some examples when the outer coil 712 and the inner coil 714 have the same pitch direction, no gaps may be visible when the coil layer 710 is bent. For example, when the coil layer 710 is bent, the ribbon elements 717 of the outer coil 712 may cover any gaps that may open between the ribbon elements 719 of the inner coil 714. Additionally, when the outer coil 712 and the inner coil 714 have the same pitch direction (e.g., the pitch direction P1), the coil layer 710 may have a higher torsional stiffness when a torque is applied to the coil layer 710 in a clockwise direction than when a torque is applied to the coil layer 710 in a counterclockwise direction. In some examples, when the outer coil 712 and the inner coil 714 have the same pitch direction (e.g., the pitch direction P2), the coil layer 710 may have a higher torsional stiffness when a torque is applied to the coil layer 710 in a counterclockwise direction than when a torque is applied to the coil layer 710 in a clockwise direction.

In some examples, the outer coil 712 and inner coil 714 may have the same pitch direction but at different pitch angles (e.g., defined by different angles relative to the longitudinal axis A). FIG. 9B illustrates a side view of the distal portion of the coil layer 710 in an alternative configuration where the outer coil 712 and inner coil 714 have opposing pitch directions. For example, the outer coil 712 is wound in the pitch direction P1, and the inner coil 714 is wound in an opposing pitch direction P2. In other examples, the outer coil 712 may be wound in the pitch direction P2, and the inner coil 714 may be wound in the pitch direction P1.

When the outer coil 712 and inner coil 714 have opposing pitch directions, the elongate device 150 may be better able to accommodate tight bending (e.g., a small radius of curvature). For example, when the outer coil 712 and inner coil 714 have opposing pitch directions, the outer coil 712 and inner coil 714 may be able to bend to a tighter radius of curvature than a single coil is able to bend or than two coils with the same pitch direction are able to bend without causing the material of the outer coil 712 and inner coil 714 to be permanently deformed. Having the outer coil 712 and inner coil 714 include opposing pitch directions may also help prevent one or more ribbon elements 719 of the inner coil 714 from being stuck in a gap 713 between two or more ribbon elements 717 of the outer coil 712 when the elongate device 150 is bent.

In some examples, the outer coil 712 and/or inner coil 714 includes multiple ribbons that are interwoven. As shown in FIG. 9C, the outer coil 712 includes two ribbons 712A, 712B that are interwoven. Each of the ribbons 712A, 712B is around the inner coil 714. In this example, the ribbons 712A and 712B each have the pitch direction P1 while the inner coil 714 has the opposite pitch direction P2. In another example, the ribbon for the inner coil 714 and outer coil 712 may have the same pitch direction, P1 or P2. When the outer coil 712 includes two (or more) ribbons 712A, 712B, the number of available bonding locations 720 between the outer coil 712 and the inner coil 714 may increase. In some examples, the inner coil 714 includes multiple ribbons.

As shown in FIGS. 9A-9C, each of the outer coil 712 and inner coil 714 may have a consistent ribbon width along the length of the coil layer 710. For example, a ribbon width W1 of the outer coil 712 may remain consistent from the proximal portion 716 of the coil layer 710 to the distal portion 718 of the coil layer 710. In some examples, the ribbon width of the inner coil 714 is the same as the ribbon width W1 of the outer coil 712. Alternatively, the ribbon widths of the outer coil 712 and inner coil 714 may be different. In some examples, the ribbon width W1 of the outer coil 712 and/or the ribbon width of the inner coil 714 varies along the length of the coil layer 710, as discussed above with respect to the coils of the coil layer 310. For example, the ribbon width of the outer coil 712 may be wider at the proximal portion 716 than at the distal portion 718. Additionally, or alternatively, the ribbon width of the inner coil 714 may be wider at the proximal portion 716 than at the distal portion 718. One or both of the outer coil 712 and inner coil 714 may include any other configuration of variable ribbon width (e.g., wider at the distal portion 718 than at the proximal portion 716, wider in the middle of the coil layer 710 than at the proximal and distal portions 716, 718, or any other configuration). In some examples when a coil of the coil layer 710 has a variable ribbon width, the coil layer 710 may have variable bending stiffness as a result of the variable ribbon width.

In some examples, one or both of the outer coil 712 and inner coil 714 may be closed pitch. When a coil is closed pitch, there is no gap between the ribbon elements of the coil when the coil layer 710 is unbent. In examples when both the outer coil 712 and inner coil 714 are closed pitch, the bonding locations 720 may be on the ribbon elements 717 of the outer coil 712 and the ribbon elements 719 of the inner coil 714 themselves. When a coil is open pitch, there is a gap between the ribbon elements of the coil.

In some examples, one or both of the outer coil 712 and inner coil 714 may be single filar wires (see FIGS. 9A and 9B). In some examples, one or both of the outer coil 712 and inner coil 714 may be multi-filar wires (see the outer coil 712 in FIG. 9C, for example). In some examples, the ribbon(s) of one or both of the outer coil 712 and inner coil 714 may be formed from a round wire. In some examples, the ribbon(s) of one or both of the outer coil 712 and inner coil 714 may be formed from a flat wire. Any combination of single filar wires, multi-filar wires, round wires, flat wires, open pitch coils, closed pitch coils, variable ribbon width, and consistent ribbon width may be used in the coil layer 710.

In some examples, the ribbon(s) of one or both of the outer coil 712 and inner coil 714 may be formed from a hypotube. The hypotube may be laser cut to create slits that define a helical winding pattern in the hypotube. In some examples, the hypotube may be water jet cut to create a helical winding pattern in the hypotube.

The outer coil 712 and inner coil 714 may be metal, polymer, or any other suitable material. The bonding technique used to bond the outer coil 712 and inner coil 714 at the bonding locations 720 may be any type of appropriate bonding technique that is suitable to the specific materials used for the outer coil 712 and inner coil 714. For example, if the outer coil 712 and inner coil 714 are metal, the outer coil 712 and inner coil 714 may be welded together, with each bond being a welding location. The type of weld(s) used to weld the outer coil 712 and inner coil 714 together may be any one or more of a spot weld, an electroresistive weld, a seam weld, a laser weld, any combination thereof, and/or any other type of weld. Additionally, or alternatively, the outer coil 712 and inner coil 714 may be bonded together with an adhesive or other bonding material at the bonding locations 720.

FIG. 10 illustrates a welding mechanism 800 according to some examples. In some examples, the welding mechanism 800 may be used for electroresistive welding to form bonds in the coil layer 710. The welding mechanism 800 includes a mandrel 810, which may be a conductive mandrel, one or more guide wheels 820, and one or more weld wheels 830, which may be seam weld wheels. The guide wheels 820 may include a groove 822 that is sized and shaped to receive the mandrel 810. In some examples, the guide wheels 820 may be made of a non-conductive material. The weld wheels 830 may include an outer surface 832 that contacts the mandrel 810 or anything fit over the mandrel 810, such as the coil layer 710. The weld wheels 830 are made of a conductive material. While two guide wheels 820 are shown in FIG. 10, the welding mechanism 800 may include any other number of guide wheels 820. While two weld wheels 830 are shown in FIG. 10, the welding mechanism 800 may include any other number of weld wheels 830.

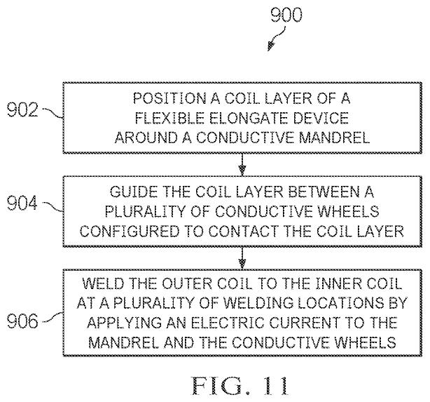

The operation of the welding mechanism 800 will be discussed in greater detail below with reference to FIG. 11. FIG. 11 is a flowchart illustrating a method 900 of welding a coil layer (e.g., the coil layer 710 or the coil layer 310) of a flexible elongate device (e.g., the flexible elongate device 150). The method 900 will be discussed with reference to FIG. 10. The method 900 is illustrated as a set of operations or processes 902 through 906. The processes illustrated in FIG. 11 may be performed in a different order than the order shown in FIG. 11, and one or more of the illustrated processes might not be performed in some examples of the method 900. Additionally, one or more processes that are not expressly illustrated in FIG. 11 may be included before, after, in between, or as part of the illustrated processes.

At a process 902, a coil layer (e.g., the coil layer 710 or the coil layer 310) of a flexible elongate device (e.g., the flexible elongate device 150) is positioned around a conductive mandrel (e.g., the conductive mandrel 810). As shown in FIG. 10, the inner coil 714 may be positioned around the mandrel 810, and the outer coil 712 may be positioned around the inner coil 714.

At a process 904, the coil layer 710 is guided between a plurality of conductive wheels (e.g., the weld wheels 830). The weld wheels 830 contact the coil layer 710 when the coil layer 710 is guided between the weld wheels 830. For example, and outer circumferential surface 832 of the weld wheels 830 may contact the coil layer 710. In some examples, the mandrel 810 and the coil layer 710 may be guided together in the direction D1 between the weld wheels 830. The coil layer 710 may be temporarily coupled to the mandrel 810, such as via an adhesive connection and/or a mechanical connection. The coil layer 710 may be removed from the mandrel 810 after the outer coil 712 and inner coil 714 are welded together.

In some examples, the coil layer 710 is also guided between the guide wheels 820. The guide wheels 820 contact the coil layer 710 when the coil layer 710 is guided between the guide wheels 820. The coil layer 710 and/or the mandrel 810 may be received by the grooves 822 of the guide wheels 820 when the coil layer 710 and/or the mandrel 810 are guided between the guide wheels 820. In some examples, the guide wheels 820 help position the coil layer 710 and/or the mandrel 810 so that the coil layer 710 is guided between the weld wheels 830.

In some examples, as shown in FIG. 10, the weld wheels 830 are equally spaced apart around a circumference of the coil layer 710. For example, the weld wheels 830 may be spaced 180° apart around the circumference of the coil layer 710. Alternatively, the weld wheels 830 may be non-equally spaced around the circumference of the coil layer 710. In examples when the weld wheels 830 are equally spaced around the circumference of the coil layer 710, the forces applied to the coil layer 710 by the weld wheels 830 may cancel each other out and may obviate the need for additional external support to support the coil layer 710 as it travels between the weld wheels 830. In examples when the weld wheels 830 are non-equally spaced around the circumference of the coil layer 710, the forces applied to the coil layer 710 by the weld wheels 830 may not cancel each other out, and additional external support to support the coil layer 710 as it travels between the weld wheels 830 may be needed.

At a process 906, the outer coil 712 is welded to the inner coil 714 at a plurality of welding locations (e.g., the bonding locations 720) by applying an electric current between the mandrel 810 and the weld wheels 830. The electric current may be applied between the weld wheels 830 and the mandrel 810 at different times as the coil layer 710 is guided between the weld wheels 830. Each time the electric current is provided, a bond 725 (e.g., a spot/seam weld) is placed between the outer coil 712 and inner coil 714 bonding the outer coil 712 and inner coil 714 together at that bonding location 720. In some examples, the weld wheels 830 are pressed radially inward in a direction D2 toward the longitudinal axis A of the coil layer 710. Pressing the weld wheels 830 radially inward increases a contact pressure between the weld wheels 830 and the coil layer 710. The increased contact pressure may result in a stronger bond 725 between the outer coil 712 and inner coil 714 when the electric current is applied between the weld wheels 830 and the mandrel 810.

In the following description, specific details describe some examples consistent with the present disclosure. Numerous specific details are set forth in order to provide a thorough understanding of the examples. It will be apparent to one skilled in the art, however, that some examples may be practiced without some or all of these specific details. The specific examples disclosed herein are meant to be illustrative but not limiting. One skilled in the art may realize other elements that, although not specifically described, are within the scope and the spirit of this disclosure. In addition, to avoid unnecessary repetition, one or more features shown and described in association with one example may be incorporated into other examples unless specifically described otherwise or if the one or more features would make an example non-functional. In some instances, well known methods, procedures, components, and circuits have not been described in detail so as not to unnecessarily obscure aspects of the examples.

In some examples, the components discussed below may be part of a robotic-assisted system as described in further detail below. The robotic-assisted system may be suitable for use in, for example, surgical, robotic-assisted surgical, diagnostic, therapeutic, or biopsy procedures. While some examples are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems and general robotic, general robotic-assisted, or robotic medical systems.

As shown in FIG. 12, a medical system 100 generally includes a manipulator assembly 102 for operating a medical instrument 104 (e.g., flexible elongate device 150) in performing various procedures on a patient P positioned on a table T. The manipulator assembly 102 may be robotic-assisted, non-robotic-assisted, or a hybrid robotic-assisted and non-robotic-assisted assembly with select degrees of freedom of motion that may be motorized and/or robotic-assisted and select degrees of freedom of motion that may be non-motorized and/or non-robotic-assisted. The medical system 100 may further include a master assembly 106, which generally includes one or more control devices for controlling manipulator assembly 102. Manipulator assembly 102 supports medical instrument 104 and may optionally include a plurality of actuators or motors that drive inputs on medical instrument 104 in response to commands from a control system 109. The actuators may optionally include drive systems that when coupled to medical instrument 104 may advance medical instrument 104 into a naturally or surgically created anatomic orifice.

Medical system 100 also includes a display system 110 for displaying an image or representation of the surgical site and medical instrument 104 generated by sub-systems of sensor system 108. Display system 110 and master assembly 106 may be oriented so operator O can control medical instrument 104 and master assembly 106 with the perception of telepresence. Additional information regarding the medical system 100 and the medical instrument 104 may be found in International Application Publication No. WO 2018/195216, filed on Apr. 18, 2018, entitled “Graphical User Interface for Monitoring an Image-Guided Procedure,” which is incorporated by reference herein in its entirety.

In some examples, medical instrument 104 may include components of an imaging system (discussed in more detail below), which may include an imaging scope assembly or imaging instrument that records a concurrent or real-time image of a surgical site and provides the image to the operator or operator O through one or more displays of medical system 100, such as one or more displays of display system 110. The concurrent image may be, for example, a two or three-dimensional image captured by an imaging instrument positioned within the surgical site. In some examples, the imaging system includes endoscopic imaging instrument components that may be integrally or removably coupled to medical instrument 104. However, in some examples, a separate endoscope, attached to a separate manipulator assembly may be used with medical instrument 104 to image the surgical site. In some examples, as described in detail below, the imaging instrument alone or in combination with other components of the medical instrument 104 may include one or more mechanisms for cleaning one or more lenses of the imaging instrument when the one or more lenses become partially and/or fully obscured by fluids and/or other materials encountered by the distal end of the imaging instrument. In some examples, the one or more cleaning mechanisms may optionally include an air and/or other gas delivery system that is usable to emit a puff of air and/or other gasses to blow the one or more lenses clean. Examples of the one or more cleaning mechanisms are discussed in more detail in International Application Publication No. WO/2016/025465, filed on Aug. 11, 2016, entitled “Systems and Methods for Cleaning an Endoscopic Instrument”; U.S. patent application Ser. No. 15/508,923, filed on Mar. 5, 2017, entitled “Devices, Systems, and Methods Using Mating Catheter Tips and Tools”; and U.S. patent application Ser. No. 15/503,589, filed Feb. 13, 2017, entitled “Systems and Methods for Cleaning an Endoscopic Instrument,” each of which is incorporated by reference herein in its entirety. The imaging system may be implemented as hardware, firmware, software or a combination thereof which interact with or are otherwise executed by one or more computer processors, which may include the processors of the control system 109.

Control system 109 includes one or more memories and one or more computer processors (not shown) for effecting control between medical instrument 104, master assembly 106, sensor system 108, and display system 110. Control system 109 also includes programmed instructions (e.g., a non-transitory machine-readable medium storing the instructions) to implement some or all of the methods described in accordance with aspects disclosed herein, including instructions for providing information to display system 110.

FIG. 13 is a diagram of a medical instrument system 200 according to some examples. Medical instrument system 200 includes elongate device 202, such as a flexible catheter (e.g., the medical instrument 104, which may be the flexible elongate device 150), coupled to a drive unit 204. Elongate device 202 includes a flexible body 216 having proximal end 217 and distal end or tip portion 218. Medical instrument system 200 further includes a tracking system 230 for determining the position, orientation, speed, velocity, pose, and/or shape of distal end 218 and/or of one or more segments 224 along flexible body 216 using one or more sensors and/or imaging devices as described in further detail below.

Tracking system 230 may optionally track distal end 218 and/or one or more of the segments 224 using a shape sensor 222. Shape sensor 222 may optionally include an optical fiber aligned with flexible body 216 (e.g., provided within an interior channel (not shown) or mounted externally). The optical fiber of shape sensor 222 forms a fiber optic bend sensor for determining the shape of flexible body 216. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. patent application Ser. No. 11/180,389, filed on Jul. 13, 2005, entitled “Fiber Optic Position and Shape Sensing Device and Method Relating Thereto”; U.S. patent application Ser. No. 12/047,056, filed on Jul. 16, 2004, entitled “Fiber-Optic Shape and Relative Position Sensing”; and U.S. Pat. No. 6,389,187, filed on Jun. 17, 1998, entitled “Optical Fibre Bend Sensor”, each of which is incorporated by reference herein in its entirety. Sensors in some examples may employ other suitable strain sensing techniques, such as Rayleigh scattering, Raman scattering, Brillouin scattering, and Fluorescence scattering. In some examples, the shape of the elongate device may be determined using other techniques. For example, a history of the distal end pose of flexible body 216 can be used to reconstruct the shape of flexible body 216 over the interval of time. In some examples, tracking system 230 may optionally and/or additionally track distal end 218 using a position sensor system 220. Position sensor system 220 may be a component of an EM sensor system with position sensor system 220 including one or more conductive coils that may be subjected to an externally generated electromagnetic field. Each coil of the EM sensor system then produces an induced electrical signal having characteristics that depend on the position and orientation of the coil relative to the externally generated electromagnetic field. In some examples, position sensor system 220 may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point. Further description of a position sensor system is provided in U.S. Pat. No. 6,380,732, filed on Aug. 11, 1999, entitled “Six-Degree of Freedom Tracking System Having a Passive Transponder on the Object Being Tracked”, which is incorporated by reference herein in its entirety.

Flexible body 216 includes a channel (not shown) sized and shaped to receive a medical instrument 226. Further description of a medical instrument received by a flexible body is provided in U.S. Provisional Patent Application No. 63/077,059 , filed on Sep. 11, 2020, entitled “Systems for Coupling and Storing an Imaging Instrument”, which is incorporated by reference herein in its entirety.

Flexible body 216 may also house cables, linkages, or other steering controls (not shown) that extend between drive unit 204 and distal end 218 to controllably bend distal end 218 as shown, for example, by broken dashed line depictions 219 of distal end 218. In some examples, at least four cables are used to provide independent “up-down” steering to control a pitch of distal end 218 and “left-right” steering to control a yaw of distal end 218. Steerable elongate devices are described in detail in U.S. patent application Ser. No. 13/274,208, filed on Oct. 14, 2011, entitled “Catheter with Removable Vision Probe”, which is incorporated by reference herein in its entirety.

The information from tracking system 230 may be sent to a navigation system 232 where it is combined with information from image processing system 231 and/or the preoperatively obtained models to provide the operator with real-time position information. In some examples, the real-time position information may be displayed on display system 110 of FIG. 12 for use in the control of medical instrument system 200. In some examples, control system 109 of FIG. 12 may utilize the position information as feedback for positioning medical instrument system 200. Various systems for using fiber optic sensors to register and display a surgical instrument with surgical images are provided in U.S. patent application Ser. No. 13/107,562, filed on May 13, 2011, entitled “Medical System Providing Dynamic Registration of a Model of an Anatomic Structure for Image-Guided Surgery,” which is incorporated by reference herein in its entirety.

In some examples, medical instrument system 200 may be robotic-assisted within medical system 100 of FIG. 12. In some examples, manipulator assembly 102 of FIG. 12 may be replaced by direct operator control. In some examples, the direct operator control may include various handles and operator interfaces for hand-held operation of the instrument.

The singular forms “a”, “an”, and “the” are intended to include the plural forms as well, unless the context indicates otherwise. And the terms “comprises,” “comprising,” “includes,” “has,” and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components. Components described as coupled may be directly or indirectly communicatively coupled. The auxiliary verb “may” likewise implies that a feature, step, operation, element, or component is optional.

In the description, specific details have been set forth describing some examples. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the following description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions.

Any alterations and further modifications to the described devices, instruments, methods, and any further application of the principles of the present disclosure are fully contemplated as would normally occur to one skilled in the art to which the disclosure relates. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The systems and methods described herein may be suited for navigation and treatment of anatomic tissues, via natural or surgically created connected passageways, in any of a variety of anatomic systems, including the lung, colon, the intestines, the kidneys and kidney calices, the brain, the heart, the circulatory system including vasculature, and/or the like. Although some of the examples described herein refer to surgical procedures or instruments, or medical procedures and medical instruments, the techniques disclosed apply to non-medical procedures and non-medical instruments. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

Further, although some of the examples presented in this disclosure discuss robotic-assisted systems or remotely operable systems, the techniques disclosed are also applicable to computer-assisted systems that are directly and manually moved by operators, in part or in whole.

Additionally, one or more elements in examples of this disclosure may be implemented in software to execute on a processor of a computer system such as a control processing system. When implemented in software, the elements of the examples of the present disclosure are essentially the code segments to perform the necessary tasks. The program or code segments can be stored in a processor readable storage medium (e.g., a non-transitory storage medium) or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In some examples, the control system may support wireless communication protocols such as Bluetooth, Infrared Data Association (IrDA), HomeRF, IEEE 802.11, Digital Enhanced Cordless Telecommunications (DECT), ultra-wideband (UWB), ZigBee, and Wireless Telemetry.

A computer is a machine that follows programmed instructions to perform mathematical or logical functions on input information to produce processed output information. A computer includes a logic unit that performs the mathematical or logical functions, and memory that stores the programmed instructions, the input information, and the output information. The term “computer” and similar terms, such as “processor” or “controller” or “control system”, are analogous.

Note that the processes and displays presented may not inherently be related to any particular computer or other apparatus, and various systems may be used with programs in accordance with the teachings herein. The required structure for a variety of the systems discussed above will appear as elements in the claims. In addition, the examples of the present disclosure are not described with reference to any particular programming language. It will be appreciated that a variety of programming languages may be used to implement the teachings of the present disclosure as described herein.

While certain example examples of the present disclosure have been described and shown in the accompanying drawings, it is to be understood that such examples are merely illustrative of and not restrictive to the broad disclosed concepts, and that the examples of the present disclosure not be limited to the specific constructions and arrangements shown and described, since various other modifications may occur to those ordinarily skilled in the art.

1. A flexible elongate device, comprising:
an articulable distal section; and
a non-articulable proximal section that is proximal to the articulable distal section,
wherein a distal portion of the non-articulable proximal section has a first bending stiffness and a proximal portion of the non-articulable proximal section has a second bending stiffness, the first bending stiffness being less than the second bending stiffness.

2. The flexible elongate device of claim 1, wherein the distal portion of the non-articulable proximal section includes a first subset of bonds having a first spatial density, wherein the proximal portion of the non-articulable proximal section includes a second subset of bonds having a second spatial density, and wherein the first spatial density is less than the second spatial density.

3. The flexible elongate device of claim 2, wherein the first spatial density is less than the second spatial density along an axial length of the flexible elongate device.

4. The flexible elongate device of claim 1, wherein the non-articulable proximal section includes a coil layer including:
an outer coil;
an inner coil within the outer coil; and
bonds between the inner coil and the outer coil that couple the inner and outer coils to each other.

5. The flexible elongate device of claim 4, wherein each of the outer coil and the inner coil are metal coils, and wherein the bonds are welds that couple the inner and outer coils to each other.

6. The flexible elongate device of claim 4, wherein a ribbon width of each of the outer and inner coils is uniform along an axial length of the coil layer.

7. The flexible elongate device of claim 4, wherein the outer coil includes a first ribbon and a second ribbon, wherein the first and second ribbons are intertwined.

8. The flexible elongate device of claim 4, wherein the outer coil has a first pitch direction, and the inner coil has a second pitch direction that is opposite the first pitch direction.

9. The flexible elongate device of claim 4, wherein the outer coil and the inner coil include one of a round wire or a flat wire.

10. The flexible elongate device of claim 4, wherein at least one of the outer coil and the inner coil includes a single filar wire or a multi-filar wire.

11. The flexible elongate device of claim 4, wherein each of the outer coil and the inner coil are closed pitch or open pitch.

12. The flexible elongate device of claim 1, wherein:
the non-articulable proximal section includes a coil;
the coil at the distal portion of the non-articulable proximal section includes a first ribbon element having a first ribbon width; and
the coil at the proximal portion of the non-articulable proximal section includes a second ribbon element having a second ribbon width that is wider than the first ribbon width.

13. The flexible elongate device of claim 1, wherein the non-articulable proximal section includes a coil layer including a plurality of coils, the plurality of coils including a first coil having variable ribbon width.

14. The flexible elongate device of claim 13, wherein the plurality of coils of the coil layer further includes:
a second coil having a pitch direction that is opposite a pitch direction of the first coil; and
a third coil having a pitch direction that is opposite the pitch direction of the second coil,
wherein the third coil includes a ribbon element having a uniform ribbon width along an axial length of the coil layer, and
wherein the pitch direction of the third coil is equal to the pitch direction of the first coil.

15. A flexible elongate device, comprising:
a coil layer that provides variable bending stiffness for the flexible elongate device along an axial length of the coil layer, the coil layer including a plurality of coils, the plurality of coils including a first coil including ribbon elements having variable ribbon width, wherein:
in a first region along the axial length of the coil layer having a first bending stiffness, a first ribbon element of the first coil has a first ribbon width; and
in a second region along the axial length of the coil layer having a second bending stiffness that is higher than the first bending stiffness, a second ribbon element of the first coil has a second ribbon width that is wider than the first ribbon width.

16. The flexible elongate device of claim 15, wherein the plurality of coils of the coil layer further includes a second coil.

17. The flexible elongate device of claim 16, wherein the second coil includes a uniform ribbon width along an axial length of the coil layer.

18. The flexible elongate device of claim 16, wherein the second coil is a braided coil.

19. The flexible elongate device of claim 16, wherein the plurality of coils of the coil layer further includes a third coil.

20. The flexible elongate device of claim 15, wherein adjacent coils of the plurality of coils have opposite pitch directions.

21-82. (canceled)
